SEQUENCE LISTING (6 sequences)

SEQ ID NO: 1 - DNA, 461 bp, Artificial Sequence
  amplified fragment of pTaU6-gRNA-C5 vector
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggcgcatagtgagatgcagcccacgtgagttcagcaacggtctagcgctgggcttttaggcccgcatgatcgggctttgtcgggtggtcgacgtgttcacgattggggagagcaacgcagcagttcctcttagtttagtcccacctcgcctgtccagcagagttctgaccggtttataaactcgcttgctgcatcagacttgttgccgtaggtgcccgggttttagagctagaaatagcaagttaaaataaggctagtccgttatcaacttgaaaaagtggcaccgagtcggtgcttttttt

SEQ ID NO: 2 - DNA, 5713 bp, Artificial Sequence
  full-length sequence of the pJIT163-2NLSCas9 vector
cctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcttccaccatggcgtgcaggtcgactctagaggatccatggctcctaagaagaagcggaaggttggtattcacggggtgcctgcggctatggataagaagtacagcattggtctggacatcgggacgaattccgttggctgggccgtgatcaccgatgagtacaaggtcccttccaagaagtttaaggttctggggaacaccgatcggcacagcatcaagaagaatctcattggagccctcctgttcgactcaggcgagaccgccgaagcaacaaggctcaagagaaccgcaaggagacggtatacaagaaggaagaataggatctgctacctgcaggagattttcagcaacgaaatggcgaaggtggacgattcgttctttcatagattggaagaaagtttcctcgtcgaggaagataagaagcacgagaggcatcctatctttggcaacattgtcgacgaggttgcctatcacgaaaagtaccccacaatctatcatctgcggaagaagcttgtggactcgactgataaggcggaccttagattgatctacctcgctctggcacacatgattaagttcaggggccattttctgatcgagggggatcttaacccggacaatagcgatgtggacaagttgttcatccagctcgtccaaacctacaatcagctctttgaggaaaacccaattaatgcttcaggcgtcgacgccaaggcgatcctgtctgcacgcctttcaaagtctcgccggcttgagaacttgatcgctcaactcccgggcgaaaagaagaacggcttgttcgggaatctcattgcactttcgttggggctcacaccaaacttcaagagtaattttgatctcgctgaggacgcaaagctgcagctttccaaggacacttatgacgatgacctggataaccttttggcccaaatcggcgatcagtacgcggacttgttcctcgccgcgaagaatttgtcggacgcgatcctcctgagtgatattctccgcgtgaacaccgagattacaaaggccccgctctcggcgagtatgatcaagcgctatgacgagcaccatcaggatctgacccttttgaaggctttggtccggcagcaactcccagagaagtacaaggaaatcttctttgatcaatccaagaacggctacgctggttatattgacggcggggcatcgcaggaggaattctacaagtttatcaagccaattctggagaagatggatggcacagaggaactcctggtgaagctcaatagggaggaccttttgcggaagcaaagaactttcgataacggcagcatccctcaccagattcatctcggggagctgcacgccatcctgagaaggcaggaagacttctacccctttcttaaggataaccgggagaagatcgaaaagattctgacgttcagaattccgtactatgtcggaccactcgcccggggtaattccagatttgcgtggatgaccagaaagagcgaggaaaccatcacaccttggaacttcgaggaagtggtcgataagggcgcttccgcacagagcttcattgagcgcatgacaaattttgacaagaacctgcctaatgagaaggtccttcccaagcattccctcctgtacgagtatttcactgtttataacgaactcacgaaggtgaagtatgtgaccgagggaatgcgcaagcccgccttcctgagcggcgagcaaaagaaggcgatcgtggaccttttgtttaagaccaatcggaaggtcacagttaagcagctcaaggaggactacttcaagaagattgaatgcttcgattccgttgagatcagcggcgtggaagacaggtttaacgcctcactggggacttaccacgatctcctgaagatcattaaggataaggacttcttggacaacgaggaaaatgaggatatcctcgaagacattgtcctgactcttacgttgtttgaggatagggaaatgatcgaggaacgcttgaagacgtatgcccatctcttcgatgacaaggttatgaagcagctcaagagaagaagatacaccggatggggaaggctgtcccgcaagcttatcaatggcattagagacaagcaatcagggaagacaatccttgactttttgaagtctgatggcttcgcgaacaggaattttatgcagctgattcacgatgactcacttactttcaaggaggatatccagaaggctcaagtgtcgggacaaggtgacagtctgcacgagcatatcgccaaccttgcgggatctcctgcaatcaagaagggtattctgcagacagtcaaggttgtggatgagcttgtgaaggtcatgggacggcataagcccgagaacatcgttattgagatggccagagaaaatcagaccacacaaaagggtcagaagaactcgagggagcgcatgaagcgcatcgaggaaggcattaaggagctggggagtcagatccttaaggagcacccggtggaaaacacgcagttgcaaaatgagaagctctatctgtactatctgcaaaatggcagggatatgtatgtggaccaggagttggatattaaccgcctctcggattacgacgtcgatcatatcgttcctcagtccttccttaaggatgacagcattgacaataaggttctcaccaggtccgacaagaaccgcgggaagtccgataatgtgcccagcgaggaagtcgttaagaagatgaagaactactggaggcaacttttgaatgccaagttgatcacacagaggaagtttgataacctcactaaggccgagcgcggaggtctcagcgaactggacaaggcgggcttcattaagcggcaactggttgagactagacagatcacgaagcacgtggcgcagattctcgattcacgcatgaacacgaagtacgatgagaatgacaagctgatccgggaagtgaaggtcatcaccttgaagtcaaagctcgtttctgacttcaggaaggatttccaattttataaggtgcgcgagatcaacaattatcaccatgctcatgacgcatacctcaacgctgtggtcggaacagcattgattaagaagtacccgaagctcgagtccgaattcgtgtacggtgactataaggtttacgatgtgcgcaagatgatcgccaagtcagagcaggaaattggcaaggccactgcgaagtatttcttttactctaacattatgaatttctttaagactgagatcacgctggctaatggcgaaatccggaagagaccacttattgagaccaacggcgagacaggggaaatcgtgtgggacaaggggagggatttcgccacagtccgcaaggttctctctatgcctcaagtgaatattgtcaagaagactgaagtccagacgggcgggttctcaaaggaatctattctgcccaagcggaactcggataagcttatcgccagaaagaaggactgggatccgaagaagtatggaggtttcgactcaccaacggtggcttactctgtcctggttgtggcaaaggtggagaagggaaagtcaaagaagctcaagtctgtcaaggagctcctgggtatcaccattatggagaggtccagcttcgaaaagaatccgatcgattttctcgaggcgaagggatataaggaagtgaagaaggacctgatcattaagcttccaaagtacagtcttttcgagttggaaaacggcaggaagcgcatgttggcttccgcaggagagctccagaagggtaacgagcttgctttgccgtccaagtatgtgaacttcctctatctggcatcccactacgagaagctcaagggcagcccagaggataacgaacagaagcaactgtttgtggagcaacacaagcattatcttgacgagatcattgaacagatttcggagttcagtaagcgcgtcatcctcgccgacgcgaatttggataaggttctctcagcctacaacaagcaccgggacaagcctatcagagagcaggcggaaaatatcattcatctcttcaccctgacaaaccttggggctcccgctgcattcaagtattttgacactacgattgatcggaagagatacacttctacgaaggaggtgctggatgcaacccttatccaccaatcgattactggcctctacgagacgcggatcgacttgagtcagctcggtggcgataagagacccgcagcaaccaagaaggcagggcaagcaaagaagaagaagtgacaattcgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatctttcaagatcaaaactagttccctcacaccg

SEQ ID NO: 3 - DNA, 1605 bp, Artificial Sequence
  mutant TaMLO-A gene
atggcggaggacgacgggtaccccccggcgcggacgctgccggagacgccgtcctgggcggtggcgctggtcttcgccgtcatgatcatcgtctccgtcctcctggagcacgcgctccacaagctcggccagtggttccacaagcggcacaagaacgcgctggcggaggcgctggagaagatgaaggcggagctgatgctggtgggattcatctcgctgctgctcgccgtcacgcaggacccaatctccgggatatgcatctcccagaaggccgccagcatcatgcgcccctgcaaggtggaacccggttccgtcaagagcaagtacaaggactactactgcgccaaagagggcaaggtggcgctcatgtccacgggcagcctgcaccagctccacatattcatcttcgtgctagccgtcttccatgtcacctacagcgtcatcatcatggctctaagccgtctcaagatgagaacatggaagaaatgggagacagagaccgcctccttggaataccagttcgcaaatgatcctgcgcggttccgcttcacgcaccagacgtcgttcgtgaagcggcacctgggcctgtccagcacccccggcgtcagatgggtggtggccttcttcaggcagttcttcaggtcggtcaccaaggtggactacctcaccttgagggcaggcttcatcaacgcgcacttgtcgcagaacagcaagttcgacttccacaagtacatcaagaggtccatggaggacgacttcaaagtcgtcgttggcatcagcctcccgctgtgggctgtggcgatcctcaccctcttccttgatatcgacgggatcggcacactcacctgggtttctttcatccctctcatcatcctcttgtgtgttggaaccaagctagagatgatcatcatggagatggccctggagatccaggaccggtcgagcgtcatcaagggggcacccgtggtcgagcccagcaacaagttcttctggttccaccgccccgactgggtcctcttcttcatacacctgacgctgttccagaacgcgtttcagatggcacatttcgtgtggacagtggccacgcccggcttgaaggactgcttccatatgaacatcgggctgagcatcatgaaggtcgtgctggggctggctctccagttcctgtgcagctacatcaccttccccctctacgcgctagtcacacagatgggatcaaacatgaagaggtccatcttcgacgagcagacagccaaggcgctgaccaactggcggaacacggccaaggagaagaagaaggtccgagacacggacatgctgatggcgcagatgatcggcgacgcaacacccagccgaggcacgtccccgatgcctagccggggctcatcgccggtgcacctgcttcagaagggcatgggacggtctgacgatccccagagcgcaccgacctcgccaaggaccatggaggaggctagggacatgtacccggttgtggtggcgcatcctgtacacagactaaatcctgctgacaggagaaggtcggtctcttcatcagccctcgatgccgacatccccagcgcagatttttccttcagccagggatga

SEQ ID NO: 4 - DNA, 1605 bp, Artificial Sequence
  mutant TaMLO-B gene
atggcggaggacgacgggtaccccccagcgaggacgctgccggagacgccgtcctgggcggtggccctcgtcttcgccgtcatgatcatcgtgtccgtcctcctggagcacgcgctccataagctcggccagtggttccacaagcggcacaagaacgcgctggcggaggcgctggagaagatcaaggcggagctcatgctggtgggcttcatctcgctgctgctcgccgtgacgcaggaccccatctccgggatatgcatctccgagaaggccgccagcatcatgcggccctgcaagctgccccctggctccgtcaagagcaagtacaaagactactactgcgccaaacagggcaaggtgtcgctcatgtccacgggcagcttgcaccagctgcacatattcatcttcgtgctcgccgtcttccatgtcacctacagcgtcatcatcatggctctaagccgtctcaagatgagaacctggaagaaatgggagacagagaccgcctccctggaataccagttcgcaaatgatcctgcgcggttccgcttcacgcaccagacgtcgttcgtgaagcggcacctgggcctctccagcacccccggcgtcagatgggtggtggccttcttcaggcagttcttcaggtcggtcaccaaggtggactacctcaccttgagggcaggcttcatcaacgcgcatttgtcgcataacagcaagttcgacttccacaagtacatcaagaggtccatggaggacgacttcaaagtcgtcgttggcatcagcctcccgctgtggtgtgtggcgatcctcaccctcttccttgacattgacgggatcggcacgctcacctggatttctttcatccctctcgtcatcctcttgtgtgttggaaccaagctggagatgatcatcatggagatggccctggagatccaggaccgggcgagcgtcatcaagggggcgcccgtggttgagcccagcaacaagttcttctggttccaccgccccgactgggtcctcttcttcatacacctgacgctattccagaacgcgtttcagatggcacatttcgtgtggacagtggccacgcccggcttgaagaaatgcttccatatgcacatcgggctgagcatcatgaaggtcgtgctggggctggctcttcagttcctctgcagctatatcaccttcccgctctacgcgctcgtcacacagatgggatcaaacatgaagaggtccatcttcgacgagcagacggccaaggcgctgacaaactggcggaacacggccaaggagaagaagaaggtccgagacacggacatgctgatggcgcagatgatcggcgacgcgacgcccagccgaggggcgtcgcccatgcctagccggggctcgtcgccagtgcacctgcttcacaagggcatgggacggtccgacgatccccagagcacgccaacctcgccaagggccatggaggaggctagggacatgtacccggttgtggtggcgcatccagtgcacagactaaatcctgctgacaggagaaggtcggtctcgtcgtcggcactcgatgtcgacattcccagcgcagatttttccttcagccagggatga

SEQ ID NO: 5 - DNA, 1605 bp, Artificial Sequence
  mutant TaMLO-D gene
atggcggaggacgacgggtaccccccggcgcggacgctgccggagacgccgtcctgggcggtggcgctcgtcttcgccgtcatgatcatcgtgtccgtcctcctggagcacgcgctccacaagctcggccagtggttccacaagcggcacaagaacgcgctggcggaggcgctggagaagatcaaagcggagctgatgctggtggggttcatctcgctgctgctcgccgtgacgcaggacccaatctccgggatatgcatctccgagaaggccgccagcatcatgcggccctgcagcctgccccctggttccgtcaagagcaagtacaaagactactactgcgccaaaaagggcaaggtgtcgctaatgtccacgggcagcttgcaccagctccacatattcatcttcgtgctcgccgtcttccatgtcacctacagcgtcatcatcatggctctaagccgtctcaagatgaggacatggaagaaatgggagacagagaccgcctccttggaataccagttcgcaaatgatcctgcgcggttccgcttcacgcaccagacgtcgttcgtgaagcgtcacctgggcctctccagcacccccggcatcagatgggtggtggccttcttcaggcagttcttcaggtcggtcaccaaggtggactacctcaccctgagggcaggcttcatcaacgcgcatttgtcgcataacagcaagttcgacttccacaagtacatcaagaggtccatggaggacgacttcaaagtcgtcgttggcatcagcctcccgctgtggtgtgtggcgatcctcaccctcttccttgatattgacgggatcggcacgctcacctggatttctttcatccctctcgtcatcctcttgtgtgttggaaccaagctggagatgatcatcatggagatggccctggagatccaggaccgggcgagcgtcatcaagggggcgcccgtggttgagcccagcaacaagttcttctggttccaccgccccgactgggtcctcttcttcatacacctgacgctgttccagaatgcgtttcagatggcacatttcgtctggacagtggccacgcccggcttgaagaaatgcttccatatgcacatcgggctgagcatcatgaaggtcgtgctggggctggctcttcagttcctctgcagctatatcaccttcccgctctacgcgctcgtcacacagatgggatcaaacatgaagaggtccatcttcgacgagcagacggccaaggcgctgacaaactggcggaacacggccaaggagaagaagaaggtccgagacacggacatgctgatggcgcagatgatcggcgacgcgacgcccagccgaggggcgtcgcccatgcctagccggggctcgtcgccagtgcacctgcttcacaagggcatgggacggtccgacgatccccagagcacgccaacctcgccaagggccatggaggaggctagggacatgtacccggttgtggtggcgcatccagtgcacagactaaatcctgctgacaggagaaggtcggtctcttcgtcggcactcgatgccgacatccccagcgcagatttttccttcagccagggatga

SEQ ID NO: 6 - DNA, 8111 bp, Artificial Sequence
  sequence of the TALENs (T-MLO)
tcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgatcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcatctagaatggtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgccggatcccagctggtgaagtccgagctggaagaaaaaaagagcgagctgcgccacaagctcaagtacgtgccccacgagtacatcgagctgatcgagatcgcccgcaacagcacccaagaccgcatcctggagatgaaagtgatggagttcttcatgaaggtgtacggctaccgcggcaagcacctgggcggctcccgcaagcccgatggcgccatctacaccgtgggctcccccatcgactatggcgtcattgtcgacaccaaggcctactccggcggctacaacttacccatcggtcaggccgacgagatgcaacgctacgtgaaggagaaccagacccgcaataagcacattaatcccaacgagtggtggaaggtgtacccctcctccgtgaccgagttcaaattcctgttcgtgtccggccacttcaagggcaattataaggcccaactgacccgcctgaaccacaagaccaactgcaacggcgccgtgctgtccgtggaggaactgctgatcggcggcgagatgatcaaggctggtaccctgaccctggaagaggtgcgccgcaagttcaacaatggtgaaatcaatttcaggtccggcggcggagagggcagaggaagtcttctaacatgcggtgacgtggaggagaatcccggccctaggatggactacaaagaccatgacggtgattataaagatcatgacatcgattacaaggatgacgatgacaagatggcccccaagaagaagaggaaggtgggcattcacggggtgccggctagcatggtggatctacgcacgctcggctacagtcagcagcagcaagagaagatcaaaccgaaggtgcgttcgacagtggcgcagcaccacgaggcactggtgggccatgggtttacacacgcgcacatcgttgcgctcagccaacacccggcagcgttagggaccgtcgctgtcacgtatcagcacataatcacggcgttgccagaggcgacacacgaagacatcgttggcgtcggcaaacagtggtccggcgcacgcgccctggaggccttgctcacggatgcgggggagttgagaggtccgccgttacagttggacacaggccaacttgtgaagattgcaaaacgtggcggcgtgaccgcaatggaggcagtgcatgcatcgcgcaatgcactgacgggtgcccccctgaacctgaccccggaccaagtggtggctatcgccagcaacaagggcggcaagcaagcgctcgaaacggtgcagcggctgttgccggtgctgtgccaggaccatggcctgaccccggaccaagtggtggctatcgccagcaacaagggcggcaagcaagcgctcgaaacggtgcagcggctgttgccggtgctgtgccaggaccatggcctgaccccggaccaagtggtggctatcgccagcaacaagggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacaagggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaagggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacaagggcggcaagcaagcgctcgaaagcattgtggcccagctgagccggcctgatccggcgttggccgcgttgaccaacgaccacctcgtcgccttggcctgcctcggcggacgtcctgccatggatgcagtgaaaaagggattgccgcacgcgccggaattgatcagaagagtcaatcgccgtattggcgaacgcacgtcccatcgcgttgccagatctcaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatggagcgatatgtcgaagaaaatcaaacacgaaacaaacatctcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtgagacggaaatttaataacggcgagataaacttttaataggaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttact

(19) United States
(12) Patent Application Publication
(10) Pub. No.: US 2018/0073035 A1
(43) Pub. Date: Mar. 15, 2018

(54) A METHOD FOR PRECISE MODIFICATION OF PLANT VIA TRANSIENT GENE EXPRESSION

(71) Applicant: Institute of Genetics and Developmental Biology, Chinese Academy of Sciences, Beijing (CN)

(72) Inventors: Caixia Gao, Beijing (CN); Yanpeng Wang, Beijing (CN); Yi Zhang, Beijing (CN); Jinxing Liu, Beijing (CN); Kang Zhang, Tianjin (CN)

(73) Assignee: Institute of Genetics and Developmental Biology, Chinese Academy of Sciences, Beijing (CN)

(21) Appl. No.: 15/544,387

(22) PCT Filed: Jan. 19, 2016
(86) PCT No.: PCT/CN2016/071352

§ 371 Date: Jul. 18, 2017

(30) Foreign Application Priority Data
Jan. 19, 2015 (CN) 201510025857.3

(51) Int. Cl. C12N 15/82; C12N 5/14; A01H 1/04; A01H 4/00

(52) U.S. Cl. CPC C12N 15/8213; A01H 4/008; A01H 1/04; C12N 5/14

(57) ABSTRACT

Provided is a method for conducting site-specific modification in a plant through gene transient expression, comprising the following steps: transiently expressing a sequence-specific nuclease specific to the target fragment in the cell or tissue of the plant of interest, wherein the sequence-specific nuclease is specific to the target site and the target site is cleaved by the nuclease, thereby the site-specific modification of the target site is achieved through DNA repairing of the plant.

### CROSS-REFERENCE TO RELATED APPLICATIONS

This application is a U.S. National Phase of International Patent Application No. PCT/CN2016/071352, filed on Jan. 19, 2016, which published as WO2016/116032 A1 on Jul. 28, 2016, and claims priority to Chinese Patent Application No. 201510025857.3, filed on Jan. 19, 2015, all of which are herein incorporated by reference in their entirety.

### SEQUENCE LISTING

The instant application contains a Sequence Listing which has been submitted in ASCII format via EFS-Web and is hereby incorporated by reference in its entirety. Said ASCII copy, created on Jun. 29, 2017, is named P20161508_sequence listing.txt, and is 25,161 bytes in size.

### TECHNICAL FIELD

The present invention belongs to the field of plant genetic engineering, and is related to a method for precise modification of plant via transient gene expression. Specifically, the invention is related to a method for achieving site-specific modification in a plant genome through a transient expression system, which has relatively higher bio-safety.

### TECHNICAL BACKGROUND

Conducting modification in the plant genome is the primary means for investigating plant genome functions and improving crops genetically. Currently, methods for modifying a plant genome are mainly focused on traditional cross breeding and mutagenesis breeding. Traditional cross breeding needs to be conducted for several generations, and thus is time-consuming and requires excessive work. It may also be limited by interspecies reproductive isolation and affected by undesirable gene linkage. Physical or chemical mutagenesis methods, such as radiation mutagenesis, EMS mutagenesis etc., can randomly introduce a large number of mutated sites in the genome, and the identifications of the mutated sites would be very difficult. Traditional gene targeting methods have very low efficiency (normally in the range of 10⁻⁶-10⁻⁵), and is limited to a few species like yeasts, mice etc. RNAi methods usually can not sufficiently down regulate the target genes, and the gene silencing effects will decrease or even completely vanish in the progeny. Therefore, gene silencing by RNAi is not genetically stable.

Genomic site-specific modification tools, which are novel techniques arisen in recent years, mainly include three categories of sequence specific nucleases (SSN): Zinc finger nucleases (ZFN), Transcription activator-like effector nucleases (TALEN), and Clustered regularly interspaced short palindromic repeats/CRISPR associated systems (CRISPR/Cas9). Their common feature is that they can act as an endonuclease to cleave specific DNA sequences, producing DNA double-strand break (DSB). The DSB can activate intrinsic repair mechanism of the cell, Non-homologous end joining (NHEJ) and Homologous recombination (HR), so as to repair the DNA damages. Through NHEJ, a disrupted chromosome can be reconnected, but the repair is usually not so precise and insertion or deletion of a few bases may take place at the site of disruption, which may result in frame-shift or deletion of key amino acid(s) and thus generate a gene knock-out mutant.

Through HR, when artificial homologous sequence is introduced, the homologous sequence is used as a template to conduct synthetic repair so as to generate a site-specific gene (or DNA fragment) replacement mutant or an insertion mutant. Currently, plant genome modifications by gene editing techniques have gradually been applied in some plants (e.g., rice, Arabidopsis, maize, and wheat etc.), but the effects are not satisfying. A main limiting factor is the genetic transformation of plants.

The introduction of a sequence-specific nuclease (SSN) into a plant cell is the basis for achieving gene editing. Currently, methods for introducing a sequence-specific nuclease into a plant cell are mainly conventional transgenic techniques. Integrating a sequence-specific nuclease gene into the plant chromosome using conventional transgenic techniques can achieve site-specific modification in the plant. Then, mutants without the modification tool can be obtained through segregation in the progeny. Such method is a well-recognized important method for obtaining site-specific mutant without a transgene. This method involves the integration of exogenous genes into the plant genome, and the transformation approach requires a selective marker (selective pressure) which renders the regeneration of plant relatively difficult; for modifying genes in vegetatively propagated crops such as potato, cassava and banana, it is difficult or impossible to segregate away sequence specific nuclease transgenes. For some transformation-recalcitrant plants, such as wheat, maize, soybean, and potato etc., genome modification will be more difficult. Therefore, gene editing techniques are not extensively used in plant genome modification. Besides, with respect to bio-safety, the USDA of USA evaluates products only according to the properties of the final product, which means that the genetically modified products obtained by conventional transgenic techniques using sequence specific nucleases like ZFN and TALEN etc., are not controlled under the GMO regulations; however, in European Union where GMO regulation is relatively strict, such products are still listed in the transgenic category and should be controlled. Therefore, it is necessary to develop a more efficient, practical, and safe method for plant genome modification.

Transient expression system refers to such a system: using gene delivery means, such as Agrobacterium, particle bombardment, and PEG-mediated protoplast transformation, to deliver an exogenous gene (sequence specific nuclease) into a cell (without integrating into the chromosome), and modifying the genome of a plant through the transient expression of the exogenous gene wherein the tissue culture throughout the plant regeneration process is performed without any selection pressure, which effectively increases the efficiency of the plant regeneration. The exogenous gene that is not integrated into the chromosome will be degraded by the plant cell, resulting in relatively higher bio-safety. Thus, it is easier and more appropriate to achieve plant genome modification using a transient expression system, which can facilitate the application of gene editing techniques in plants.

### SUMMARY OF THE INVENTION

The object of the invention is to provide a method for precise modification of the genome of a plant via transient gene expression.

Use of a transient expression system for conducting site-specific modification to a target site of a target gene in a plant belongs to the protection scope of the invention.

The method provided in the present invention for conducting site-specific modification to a target site of a target gene in a plant, specifically comprises the following steps: using a cell or tissue of the plant of interest as the subject for transient expression, transiently expressing a sequence-specific nuclease in a cell or tissue of the plant of interest; wherein said sequence-specific nuclease is specific to the target site and the target site is cleaved by said nuclease; thereby site-specific modification of the target site is achieved through DNA repairing in the plant.

In said method, the process for achieving the transient expression of the sequence-specific nuclease in a cell or tissue of the plant of interest may comprise the following steps: a) introducing a genetic material for expressing the sequence-specific nuclease into a cell or tissue of the plant of interest, b) culturing the cell or tissue as obtained in step a) in the absence of selection pressure, thereby the sequence-specific nuclease is transiently expressed in the cell or tissue of the plant of interest and the genetic material not integrated into the plant genome is degraded.

Said “genetic material” is a recombinant vector (e.g., a DNA plasmid) or a DNA linear fragment or RNA.

Said “selection pressure” refers to a medicament or reagent that is beneficial for the growth of transgenic plant but is lethal for transgene-free plant. Here, a transgenic plant refers to a plant with an exogenous gene integrated into the genome thereof. A transgene-free plant refers to a plant without an exogenous gene integrated into the genome thereof.

In the absence of selection pressure, the defending system of the plant will inhibit the entry of an exogenous gene and degrade the exogenous gene that has already been delivered into the plant. Therefore, when the cell or tissue as obtained in step a) is cultured in the absence of selection pressure, the exogenous gene (including any fragment of the genetic material for expressing the nuclease specific to the target site) will not be integrated into the genome of the plant, and the plant finally obtained is a transgene-free plant with site-specific modification.

In said method, the sequence-specific nuclease which is specific to the target site can be any nuclease that can achieve genome editing, such as Zinc finger nuclease (ZFN), and Transcription activator-like effector nuclease (TALENs), and CRISPR/Cas9 nuclease etc.

In one embodiment of the invention, the “sequence-specific nuclease” specifically refers to CRISPR/Cas9 nucleases. In some embodiments, the genetic material for expressing the CRISPR/Cas9 nucleases specific to a target site is specifically composed of a recombinant vector or DNA fragment for transcribing a guide RNA (or two recombinant vectors or DNA fragments for transcribing crRNA and tracrRNA respectively) and for expressing Cas9 protein; or is specifically composed of a recombinant vector or DNA fragment for transcribing a guide RNA (or two recombinant vectors or DNA fragments for transcribing crRNA and tracrRNA respectively) and a recombinant vector or DNA fragment or RNA for expressing Cas9 protein; or is specifically composed of a guide RNA (or a crRNA and a tracrRNA) and a recombinant vector or DNA fragment or RNA for expressing Cas9 protein. Said guide RNA is an RNA with a palindromic structure which is formed by partial base-pairing between crRNA and tracrRNA; said crRNA contains an RNA fragment capable of complementarily binding to the target site.

Furthermore, in the recombinant vector or DNA fragment for transcribing the guide RNA, the promoter for initiating the transcription of the coding nucleotide sequence of said guide RNA is a U6 promoter or a U3 promoter.

More specifically, the recombinant vector for expressing the guide RNA is a recombinant plasmid, which is obtained by inserting the coding nucleotide sequence of the “RNA fragment capable of complementarily binding to the target site” in forward direction between two BbsI restriction sites of plasmid pTaU6-gRNA or pTaU3-gRNA. The recombinant vector for expressing Cas9 protein is the vector pJIT163-2NLSCas9 or pJIT163-Ubi-Cas9.

In another embodiment of the invention, the “sequence-specific nuclease” is TALENs nucleases. The genetic material for expressing the sequence-specific nuclease specific to the target site may be a recombinant plasmid or DNA fragment or RNA that expresses paired TALEN proteins, wherein the TALEN protein is composed of a DNA binding domain capable of recognizing and binding to the target site, and a Fok I domain.

Further, in a “recombinant plasmid or DNA fragment for expressing the sequence-specific nuclease which is a plasmid that expresses paired TALEN proteins”, the promoter that initiate the transcription of the coding nucleotide sequence of said TALEN protein is a maize promoter Ubi-1.

More specifically, the recombinant plasmid that simultaneously expresses paired TALEN protein is a T-MLO vector.

In the case that the sequence-specific nuclease is Zinc finger nucleases (ZFN), the genetic material for expressing the sequence-specific nuclease which is specific to the target site may be a recombinant plasmid or DNA fragment or RNA that expresses paired ZFN proteins, wherein the ZFN protein is composed of a DNA binding domain capable of recognizing and binding to the target site, and a Fok I domain.

In said method, the cell is any cell that can act as a transient expression recipient and can regenerate into a whole plant through tissue culture; the tissue is any tissue that can act as a transient expression recipient and can regenerate into a whole plant through tissue culture. Specifically, the cell is a protoplast cell or suspension cell; the tissue is specifically callus, immature embryo, mature embryo, leaf, shoot apex, hypocotyl, young spike and the like.

In said method, the approach for introducing the genetic material into a plant cell or tissue is particle bombardment, Agrobacterium-mediated transformation, PEG-mediated protoplast transformation, electrode transformation, silicon carbide fiber-mediated transformation, vacuum infiltration transformation, or any other genetic delivery approach.

In said method, the site-specific modification is specifically insertion, deletion, and/or replacement in the target site (target fragment that the sequence-specific nuclease recognizes) in the plant genome. In some embodiments, the target site is within the encoding region of a target gene. In some embodiments, the target site is within the transcription regulation region of a target gene, such as a promoter. In some embodiments, the target gene could be a structural gene or a non-structural gene.

In some embodiments, said modification results in loss of function of the target gene. In some embodiments, said modification results in gain (or change) of function of the target gene.

The plant can be monocotyledon or dicotyledon, such as rice, Arabidopsis, maize, wheat, soybean, sorghum, potato, oat, cotton, cassava, banana and the like.

In one embodiment (Example 1) of the invention, the plant is wheat; the nuclease is CRISPR/Cas9; the target gene is wheat endogenous gene TaGASR7; the target site is 5′-CCGGGCACCTACGGCAAC-3; the recombinant vector for expressing the guide RNA is a recombinant plasmid that is obtained by inserting the DNA fragment as shown in 5′-CTTGTTGCCGTAGGTGCCCGG-3′ in a forward direction between two BbsI restriction sites of plasmid pTaU6-gRNA; the recombinant vector for expressing the Cas9 nuclease is specifically the vector pJIT163-2NLSCas9.

In another embodiment (Example 2) of the invention, the plant is wheat; the target gene is wheat endogenous gene TaMLO; the nuclease is TALENs nuclease; the target site is:

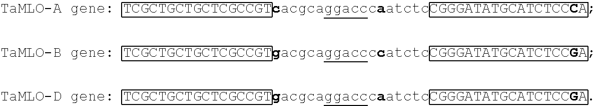

wherein the underlined region is the recognition sequence of the restriction endonuclease AvaII.

The recombinant vector for TALENs nuclease is T-MLO.

A cell or tissue, which is obtained by site-specific modification of the target site in the target gene of the plant of interest so as to allow the target gene to lose its functions or gain a function, also fall within the scope of the invention.

A modified plant regenerated from the cell or tissue of the invention also falls within the protection scope of the invention.

Further, a transgene-free plant obtained through a screen from the modified plants, which contains no integrated exogenous gene in the genome and which is genetically stable, also falls within the protection scope of the invention.

The invention also provides a method for breeding transgene-free modified plant. Specifically, the method may comprise the following steps:

(a) performing site-specific modification to a target site in a target gene of a plant of interest using the above mentioned method, so as to obtain a modified plant;

(b) obtaining a plant from the modified plant of step (a), wherein the functions of the target gene in said plant are lost or changed, the genome of said plant is free of integrated exogenous gene, and said plant is genetically stable.

By transient expression of a sequence-specific nuclease, the present invention not only increases the regeneration ability of a plant, but also allows the generated mutation to be stably transmitted to the progeny. More importantly, the mutant plant as generated is free of integrated exogenous gene and thus has relatively higher bio-safety.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the site-specific mutagenesis of wheat endogenous gene TaGASR7 (PEG4000-mediated protoplast transformation) using the gRNA:Cas9 system. Lane 1 is a marker, from bottom to top: 100, 250, 500, 750, 1000, 2000, 3000, 5000 bp respectively; lane 2 and lane 3 are BcnI restriction digestion results for PCR products of protoplast DNA, wherein the protoplast were transformed with the gRNA:Cas9 system; lane 4 is BcnI digestion result for PCR product of wild-type protoplast DNA; lane 5 is the PCR product of wild-type protoplast.

FIGS. 2A-2B show the site-specific mutagenesis of wheat endogenous gene TaGASR7 (plant obtained from transient expression system by particle bombardment) using gRNA:Cas9 system. A) is the electrophoretogram. Lane 1 is a marker, from bottom to top: 100, 250, 500, 750, 1000, 2000, 3000, 5000 bp respectively; lanes 2-9 are BcnI digestion results for detecting the mutants; lanes 5 and 6 indicate homozygous mutations; lane 10 is the result of BcnI digestion for wild-type control. B) is the sequencing results for the bands from a) that were not cleaved, indicating that insertion/deletion (indel) occurred at the target site of the TaGASR7 gene. WT represents wild-type gene sequence, “−” represents a sequence with deletion, “+” represents a sequence with insertion, the number after “−/+” represents the number of the deleted or inserted nucleotides (lowercase letter in the sequence represents the inserted nucleotide), the numbers 2-8 on the left represent 7 mutants.

FIG. 3 is a gel electrophoretogram showing the amplification of wheat TaGASR7 gene mutant using primers on the pTaU6-gRNA-C5 vector. Lane 1 is a marker, from bottom to top: 100, 250, 500, 750, 1000, 2000, 3000, 5000 bp respectively; lanes 2-24 are mutants as tested; lane 25 is the positive control (plasmid pTaU6-gRNA-C5).

FIGS. 4A-4B are gel electrophoretograms to detect the transgene-free of wheat TaGASR7 gene mutant using 2 primer sets on the pJIT163-2NLSCas9 vector. A) is the amplification result using the primer pair Cas9-1F/Cas9-1R; B) is the amplification result using the primer pair Cas9-2F/Cas9-2R. Lane 1 is a marker, from bottom to top: 100, 250, 500, 750, 1000, 2000, 3000, 5000 bp respectively; lanes 2-24 are mutants as tested; lane 25 is the positive control (plasmid pJIT163-2NLSCas9).

FIG. 5 shows the mutations in the T1 generation of the TaGASR7 mutant obtained by particle bombardment transient expression with gRNA:Cas9 system. Lane 1 is a marker, from bottom to top: 100, 250, 500, 750, 1000, 2000, 3000, 5000 bp respectively; lanes 2, 3, 4, 9, and 10 are homozygous plants resulted from segregation; lane 5 is a wild-type resulted from segregation; and lanes 6, 7, and 8 are heterozygous plants resulted from segregation.

FIG. 6A-6B show the site-specific mutagenesis of wheat endogenous gene TaMLO using TALEN system (plant obtained from transient expression system by particle bombardment). A) is the electrophoretogram. Lane 1 is a marker, from bottom to top: 100, 250, 500, 750, 1000, 2000, 3000, 5000 bp respectively; lanes 2-13 are mutants as tested, lane 14 is a positive control; and lane 15 is a negative control. B) is the sequencing results for the bands recovered from a) which were not cleaved, indicating that insertion/deletion (indel) occurred at the target site of the TaMLO gene.

FIG. 7 is a gel electrophoretogram showing the digestion results of mutants in T0-21 generation obtained by site-specific mutagenesis of wheat endogenous gene TaMLO using the transient expression system. Lanes 1-48 are digestion results of 48 T1 plants in group A and group D respectively; lane 49 is a marker. A represents TaMLO-A1 gene, D represents TaMLO-D1 gene.

FIGS. 8A-8B are gel electrophoretograms to detect the transgene-free of wheat TaMLO gene mutant using primers in the T-MLO vector specific to maize promoter Ubi-1. A) represents T0 plant. lane 1 is a marker; lanes 2-13 are the PCR amplification results of 12 T0 mutants; lane 14 is a positive control. B) represents T1 plants. Lane 1 is a marker, from bottom to top: 100, 250, 500, 750, 1000, 2000, 3000, 5000 bp respectively; lanes 2-49 are gel electrophoretogram for the PCR of 48 progeny of T0-21 mutant, and lane 50 is a positive control (plasmid T-MLO).

FIGS. 9A-9E. Transgene-free genome editing in wheat by transient expression of sequence-specific nucleases. (A) Overview of the method. The sequence-specific nuclease (SSN) plasmid is delivered into immature wheat embryos by particle bombardment. After transient expression, it is degraded, while the embryos produce calluses that can regenerate mutant seedlings. (B) Sequence of an sgRNA designed to target a site within a conserved region of exon 3 of TaGASR7 homoeologs. The outcome of PCR-RE assays analyzing 12 representative TaGASR7 mutants is shown. Lanes T0-1 to T0-12 show blots of PCR fragments amplified from independent wheat plants digested with BcnI. Lanes labeled WT1 and WT2 are PCR fragments amplified from wild-type plants with and without BcnI digestion, respectively. The bands marked by red arrowheads are caused by CRISPR-induced mutations. (C) Genotypes of 12 representative mutant plants identified by sequencing. (D) Schematic of the structure of the pGE-sgRNA vector and five primer sets used for detecting transgene-free mutants. SgRNA refers to sgRNAs targeting TaGASR7, TaNAC2, TaPIN1, TaLOX2 and TdGASR7, respectively. (E) Outcome of tests for transgene-free mutants using five primer sets in 12 representative TaGASR7 mutant plants. Lanes without bands identify transgene-free mutants. Lanes labeled WT1 and WT2 show the PCR fragments amplified from a wild-type plant and the pGE-TaGASR7 vector, respectively.

FIG. 10 shows the targeted mutations in TaGASR7, TaNAC2, TaPIN1, TaLOX2 genes in wheat protoplasts. Lanes 1 and 2: digested SSN-transformed protoplasts; lanes 3 and 4: digested and undigested wild type controls; M: marker. Sequences of SSN-induced mutations are shown on the right. The wild-type sequences are shown at the top of each sequence group. The numbers at the sides indicate the type of mutations and how many nucleotides are involved.

FIGS. 11A-11C show the outcome of PCR/RE assays for TaNAC2(A), TaPIN1(B), and TaLOX2(C) mutants.

FIGS. 12A-12B show the outcome of PCR/RE analysis of tetraploid TdGASR7 mutants in Shimai11 (A) and Yumai4 (B) with specific primers.

### DETAILED EMBODIMENTS

The experimental methods used in the following Examples are all conventional methods, unless otherwise indicated.

The materials, reagents used in the following Examples are all commercially available, unless otherwise indicated.

Expression vectors pTaU6-gRNA and pJIT163-2NLSCas9 are disclosed in “Shan, Q. et al. Targeted genome modification of crop plants using a CRISPR-Cas system. Nature Biotechnology 31:686-688, (2013)”, and can be obtained from the Institute of Genetics and Developmental Biology of the Chinese Academy of Sciences.

Expression vector pJIT163-Ubi-Cas9 is disclosed in “Wang, Y. et al. Simultaneous editing of three homoeoalleles in hexaploid bread wheat confers heritable resistance to powdery mildew. Nature Biotechnology. 32, 947-951 (2014)” and can be obtained from the Institute of Genetics and Developmental Biology of the Chinese Academy of Sciences.

The wheat variety Bobwhite is disclosed in “Weeks, J. T. et al. Rapid production of multiple independent lines of fertile transgenic wheat. Plant Physiol. 102: 1077-1084, (1993)”, and can be obtained from the Institute of Genetics and Developmental Biology of the Chinese Academy of Sciences.

Wheat TaMLO gene-targeting TALENs vector T-MLO is disclosed in “Wang, Y., Cheng, X., Shan, Q., Zhang, Y., Liu, J., Gao, C., and Qiu, J. L. (2014). Simultaneous editing of three homoeoalleles in hexaploid bread wheat confers heritable resistance to powdery mildew. Nature Biotechnology. 32, 947-951”, and can be obtained from the Institute of Genetics and Developmental Biology of the Chinese Academy of Sciences.

Solutions used in the preparation and transformation of wheat protoplast are shown in Tables 1-4.

TABLE 1
50 ml enzymolysis solution

| | The amount | | Final |
|---|---|---|---|
| | added | | Concentration |
| Cellulase R10 | 0.75 | g | 1.5% |
| Macerozyme R10 | 0.375 | g | 0.75% |
| mannitol | 5.4651 | g | 0.6M |
| 2-(N-Morpholino)ethanesulfonic acid | 0.1066 | g | 10 mM |

made up to 50 ml with double distilled water, pH adjusted to 5.7 with
KOH; incubated in 55° C. water bath for 10 min, and cooled at room
temperature before adding

| | | | |
|---|---|---|---|
| CaCl₂ | 0.0735 | g | 10 mM |
| BSA | 0.05 | g | 0.1% |

filtrated with a 0.45 μm filter

TABLE 2
500 ml W5

| | The amount | | Final | |
|---|---|---|---|---|
| | added | | Concentration | |
| NaCl | 4.5 | g | 154 | mM |
| CaCl₂ | 9.189 | g | 125 | mM |
| KCl | 0.1864 | g | 5 | mM |
| 2-(N-Morpholino)ethanesulfonic acid | 0.2132 | g | 2 | mM |

made up to 500 ml with double distilled water, pH adjusted to 5.7 with NaOH

TABLE 3
10 ml MMG solution

| | The amount | | Final | |
|---|---|---|---|---|
| | added | | Concentration | |
| Mannitol (0.8M) | 5 | ml | 0.4M | |
| MgCl₂ (1M) | 0.15 | ml | 15 | mM |
| 2-(N-Morpholino)ethanesulfonic acid | 0.2 | ml | 4 | mM |
| (200 mM) | | | | |
| Double distilled water | to 10 | ml | | |

TABLE 4
4 ml PEG solution

| | |
|---|---|
| The amount | Final |
| added | Concentration |

| | | | |
|---|---|---|---|
| PEG4000 | 1.6 | g | 40% |
| Mannitol (0.8M) | 1 | ml | 0.2M |
| CaCl₂ (1M) | 0.4 | ml | 0.1M |
| Double distilled water | to 4 | ml | |

In above Tables 1-4, % represents weight-volume percentage, g/100 ml.

The media used for wheat tissue culture include:

Hypertonic medium: MS minimal medium, 90 g/L mannitol, 5 mg/L 2,4-D, 30 g/L sucrose, and 3 g/L phytogel, pH 5.8.

Induction medium: MS minimal medium, 2 mg/L 2,4-D, 0.6 mg/L cupric sulfate, 0.5 mg/L casein hydrolysates, 30 g/L sucrose, and 3 g/L phytogel, pH 5.8.

Differentiation medium: MS minimal medium, 0.2 mg/L kinetin, 30 g/L sucrose, and 3 g/L phytogel, pH 5.8.

Rooting medium: ½ of MS minimal medium, 0.5 mg/Lethanesulfonic acid, 0.5 mg/L α-naphthylacetic acid, 30 g/L sucrose, and 3 g/L phytogel, pH 5.8.

### EXAMPLES

### Example 1. Transient Expressing CRISPR/Cas9 Nuclease by Particle Bombardment to Obtain a Transgene-Free Tagasr Mutant

I. Design of the target site: target-C5

Target-C5: 5′-CCGCCGGGCACCTACGGCAAC-3; (in the TaGASR7 gene as shown in Genbank No. EU095332, positions 248-268)

II. Preparation of pTaU6-gRNA plasmid containing C5 site

C5 is the DNA sequence for the RNA that can complementarily bind to target-C5.

The following single-stranded oligonucleotides with sticky ends (underlined) were synthesized:

C5F:
5′-CTTGTTGCCGTAGGTGCCCGG-3′;
C5R:
5′-AAACCCGGGCACCTACGGCAA-3′.

Double-stranded DNA with sticky ends was formed through oligonucleotides annealing process, and inserted between the two BbsI restriction sites in pTaU6-gRNA plasmid, resulting in pTaU6-gRNA plasmid containing C5 site. The positive plasmid was verified by sequencing. A recombinant plasmid, which was obtained by inserting the DNA fragment as shown in 5′-CTTGTTGCCGTAGGTGCCCGG-3′ in forward direction at the BbsI restriction site of pTaU6-gRNA plasmid, was positive, and was designated as pTaU6-gRNA-C5.

III. Delivering the gRNA:Cas 9 system into wheat protoplast

The pJIT163-Ubi-Cas9 vector and the pTaU6-gRNA-C5 plasmid obtained in step II were introduced into the protoplast of wheat variety Bobwhite. The specific process includes:

1. Growth of Wheat Seedling

Wheat seeds were grown in a culturing room, under 25±2° C., illuminance 1000Lx, 14-16 h light/d, for about 1-2 weeks.

2. Isolation of Protoplast

1) Tender leaves of wheat were taken, and the middle part thereof was cut into 0.5-1 mm threads using a cutter blade, placed into 0.6M of mannitol solution (using water as solvent) for 10 min in dark. The mixture was then filtrated using a filter, then placed in 50 ml enzymolysis solution for 5 h of digestion (0.5 h enzymolysis in vacuum, then 4.5 h slow shaking at 10 rmp).

Note: The temperature during enzymolysis should be kept between 20-25° C., the reaction should be carried out in the dark; and the solution should be gently shaken after the reaction so as to release the protoplasts.

2) the enzymolysis product was diluted by adding 10 ml of W5, and filtrated into a 50 ml round bottom centrifuge tube using a 75 μm Nylon filter membrane.

Note: The Nylon filter membrane should be submerged in 75% (volume percentage) ethanol, washed with water and then soaked in W5 for 2 min before use.

3) 23° C., 100 g centrifugation for 3 min, and the supernatant was discarded.

4) the pellet was suspended with 10 ml W5, placed on ice for 30 min; the protoplasts eventually formed sedimentation, and the supernatant was discarded.

5) the protoplasts were suspended by adding a proper amount of MMG solution, placed on ice until transformation.

Note: The concentration of the protoplasts needs to be determined by microscopy (×100). The amount of protoplasts was 2×10⁵/ml to 1×10⁶/ml.

3. Transformation of Wheat Protoplast

1) 10 μg pJIT163-2NLSCas9 vector and 10 μg pTaU6-gRNA-C5 plasmid were added into a 2 ml centrifuge tube. 200 μl of the protoplast obtained in above step 2 was added using a pipette and then mixed by gentle patting, kept still for 3-5 min. Then 250 μl of PEG4000 was added and mixed by gentle patting. Transformation was performed in dark for 30 min;

2) 900 μl W5 (room temperature) was added and mixed by reversing, 100 g centrifugation for 3 min, and the supernatant was discarded;

3) 1 ml W5 was added and mixed by reversing, the content was gently transferred to a 6-well plate (with pre-added 1 ml W5), and then cultured at 23° C. overnight.

IV. Using PCR/RE experiments to analyze the mutagenesis of wheat endogenous gene TaGASR7 using gRNA:Cas9 system

48 hours after the transformation of wheat protoplast, genome DNA was extracted, which was used as template for PCR/RE (Polymerase Chain Reaction/Restriction digestion) experiment analysis. At the same time, the protoplasts of wild-type wheat variety Bobwhite were used as a control. PCR/RE analysis method is based on Shan, Q. et al. Rapid and efficient gene modification in rice and Brachypodium using TALENs. Molecular Plant (2013). Since the target site (positions 248-268 of Genbank No. EU095332) of wheat endogenous gene TaGASR7 (Genbank No. EU095332) contains the recognition sequence (5′-CCSGG-3′, S represents C or G) of restriction endonuclease BcnI, and thus the restriction endonuclease BcnI was used in the experiment for conducting the PCR/RE test. Primers used in the PCR amplification were:

TaGASR7-F:
5′-GGAGGTGATGGGAGGTGGGGG-3′;
TaGASR7-R:
5′-CTGGGAGGGCAATTCACATGCCA-3′.

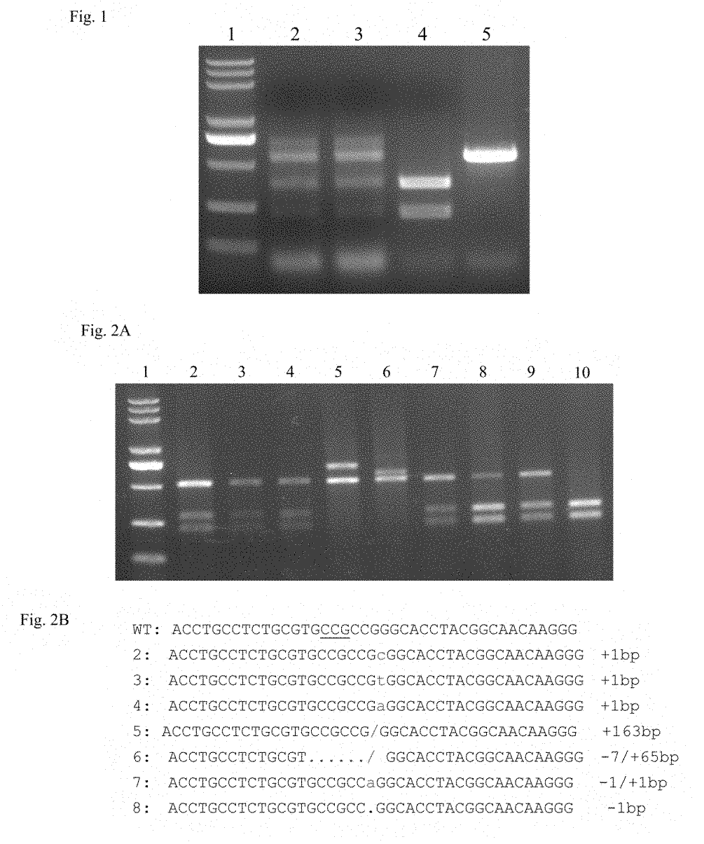

The results of PCR/RE experiments can be seen in FIG. 1, and the results showed that: mutations occurred at the target site of TaGASR7 gene, the uncut bands in the figure was recovered and sequenced, and the sequencing results showed that insertion/deletion (indel) occurred at the target site of TaGASR7 gene.

V. Site-Specific Editing of Wheat Endogenous Gene TaGASR7 Using Particle Bombardment

1) Immature embryo of the wheat variety Bobwhite was taken and treated for 4 hours using hypertonic medium;

2) A particle bombardment device was used to bombard the wheat immature embryo that was hypertonically cultured in step 1), and the pTaU6-gRNA-C5 plasmid and pJIT163-2NLSCas9 vector were introduced into the cells of the wheat immature embryo; the bombarding distance for each bombardment was 6 cm, the bombarding pressure was 1100 psi, the bombarding diameter was 2 cm, and gold powder was used in the bombardment for dispersing the DNA to be delivered; the amount of the gold powder used in each bombardment was 200 μg, and the DNA to be delivered was 0.1 μg (pTaU6-gRNA-C5 plasmid and pJIT163-2NLSCas9 vector, 0.05 μg each); and the particle size of the gold powder was 0.6 μm.

3) The wheat immature embryo bombarded in step 2) was hypertonically cultured for 16 hours;

4) The wheat immature embryo hypertonically cultured in step 3) were then sequentially subjected to 14 days of callus tissue induction culture, 28 days of differentiation culture, and 14-28 days of rooting culture, so as to obtain wheat plants.

5) DNA was extracted from the 400×4 wheat seedlings generated in step 4), and 80 mutants with gene knocked-out (site-specific) were obtained through PCR/RE tests (for specific test method and primers used, please refer to step IV). Wild-type wheat variety Bobwhite was used as control.

The test results for some of the mutants are shown in FIG. 2, and the results showed that: mutations occurred at the target site of TaGASR7 gene, the uncut bands in the figure was recovered and sequenced, and the sequencing results showed that insertion/deletion (indel) occurred at the target site of TaGASR7 gene (sequencing results can be seen in b) of FIG. 2).

6) The 80 mutants obtained in step 5) were used for PCR amplification, so as to detect whether the mutants contain fragment of the gRNA: Cas9 system plasmid. 3 pairs of primers were designed, wherein 1 pair was located in the pTaU6-gRNA-C5 vector, and 2 pairs were located in the pJIT163-2NLSCas9 vector; the DNA of the 80 mutants were used as templates, and the 3 pairs of primers were respectively used to conducting PCR amplification. Plasmid positive control (pTaU6-gRNA-C5 vector or pJIT163-2NLSCas9 vector) was also set in the experiments.

Primers in the pTaU6-gRNA-C5 vector:

U6F:
5′-GACCAAGCCCGTTATTCTGACA-3′;
CSR:
5′-AAACCCGGGCACCTACGGCAA-3′.

Theoretically, the amplified fragment should be about 382 bp, and the sequence should be positions 1-382 of SEQ ID NO:1.

Primers in the pJIT163-2NLSCas9 vector:

Cas9-1F:
5′-CCCGAGAACATCGTTATTGAGA-3′;
Cas9-1R:
5′-AACCAGGACAGAGTAAGCCACC-3′.

Theoretically, the amplified fragment should be about 1200 bp, and the sequence should be positions 3095-4264 of SEQ ID NO:2. SEQ ID NO:2 is the full-length sequence of the pJIT163-2NLSCas9 vector.

Cas9-2F:
5′-ACCAACGGTGGCTTACTCTGTC-3′;
Cas9-2R:
5′-TTCTTCTTCTTTGCTTGCCCTG-3′.

Theoretically, the amplified fragment should be about 750 bp, and the sequence should be positions 4237-4980 of SEQ ID NO:2.

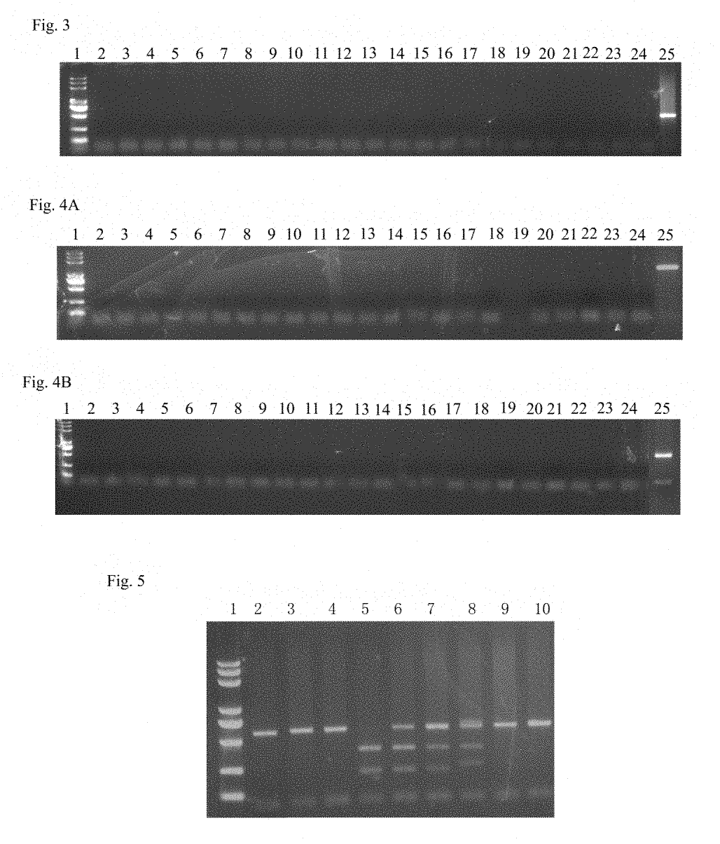

The primers in the pTaU6-gRNA-C5 vector were used to amplify wheat TaGASR7 gene mutant, and the gel electrophoretogram is shown in FIG. 3. The primers in the pJIT163-2NLSCas9 vector were used to amplify wheat TaGASR7 mutant, and the gel electrophoretogram is shown in a) of FIG. 4 (corresponding to primer pair Cas9-1F/Cas9-1R) and b) of FIG. 4 (corresponding to primer pair Cas9-2F/Cas9-2R). As can be seen from the results in FIG. 3 and FIG. 4, none of the wheat TaGASR7 mutants obtained in step 5) contained the amplified target fragment, demonstrating that the mutants did not contain fragment of the gRNA:Cas9 system plasmid. Accordingly, the present invention prevents the insertion or carrying of a transgene when performing site-specific modification in a plant, which thus avoids the transgene safety issues and public concerns.

VI. Mutant Obtained by Transient Expression of CRISPR/Cas9 System Using Particle Bombardment can be Stably Transmitted to the Progeny

T1 plants were obtained through self-fertilization of T0 mutant obtained by transient expression of CRISPR/Cas9 system using particle bombardment. TaGASR7 gene was amplified by PCR with primers. PCR products were then digested by a single enzyme BcnI (please refer to step IV). The mutations of T1 plants were examined. FIG. 5 is the PCR/RE results of 9 randomly selected T1 plants.

### Example 2. Transient Expressing TALEN Nuclease by Particle Bombardment to Obtain Inheritable and Transgene-Free Tamlo Mutant

I. Using Particle Bombardment to Transient Delivery T-MLO Vector to Perform Site-Specific Editing of Wheat MLO Gene

TELEN plasmid is the T-MLO vector, which can express paired TALEN proteins, and the TALEN protein is composed of a DNA binding domain capable of recognizing and binding to the target site, and a Fok I domain. The target sites are:

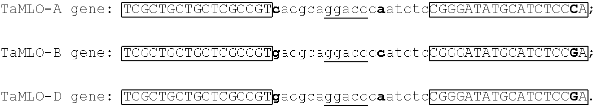

The underlined portion is the recognition sequence of restriction endonuclease AvaII.

(1) Immature embryo of the wheat variety Bobwhite was taken and hypertonically treated for 4 hours using hypertonic medium;

(2) A particle bombardment device was used to bombard the wheat immature embryo that was hypertonically cultured in step (1), and T-MLO vector was introduced into the wheat immature embryo cells; the bombarding distance for each bombardment was 6 cm, the bombarding pressure was 1100 psi, the bombarding diameter was 2 cm, and gold powder was used in the bombardment for dispersing the DNA to be delivered; the amount of the gold powder used in each bombardment was 200 μg, and the DNA to be delivered was 0.1 μg (T-MLO); and the particle size of the gold powder was 0.6 μm.

(3) The wheat immature embryo bombarded in step (2) was hypertonically cultured for 16 hours;

(4) The wheat immature embryo hypertonically cultured in step (3) were then sequentially subjected to 14 days of callus tissue induction culture, 28 days of differentiation culture, and 14-28 days of rooting culture, so as to obtain wheat plants.

(5) DNA was extracted from the wheat seedlings generated in step (4). Specific primers were used to respectively amplify TaMLO-A gene (SEQ ID NO: 3), TaMLO-B gene (SEQ ID NO: 4), and TaMLO-D gene (SEQ ID NO: 5) through PCR, and the PCR amplification products were digested by a single enzyme AvaII (since the target site of the 3 MLO genes cleaved by paired TALEN proteins all contain the recognition sequence of AvaII, accordingly, in the case a PCR product cannot be cleaved, this will indicate that a mutation occurred at that site). Wild-type wheat variety Bobwhite was used as the control.

The primer pair used for amplifying TaMLO-A gene is:

forward primer:
5′-TGGCGCTGGTCTTCGCCGTCATGATCATCGTC-3′;
reverse primer:
5′-TACGATGAGCGCCACCTTGCCCGGGAA-3′.

The primer pair used for amplifying TaMLO-B gene is:

forward primer:
5′-ATAAGCTCGGCCATGTAAGTTCCTTCCCGG-3′;
reverse primer:
5′-CCGGCCGGAATTTGTTTGTGTTTTTGTT-3′.

The primer pair used for amplifying TaMLO-D gene is:

forward primer:
5′-TGGCTTCCTCTGCTCCCTTGGTGCACCT-3′;
reverse primer:
5′-TGGAGCTGGTGCAAGCTGCCCGTGGACATT-3′.

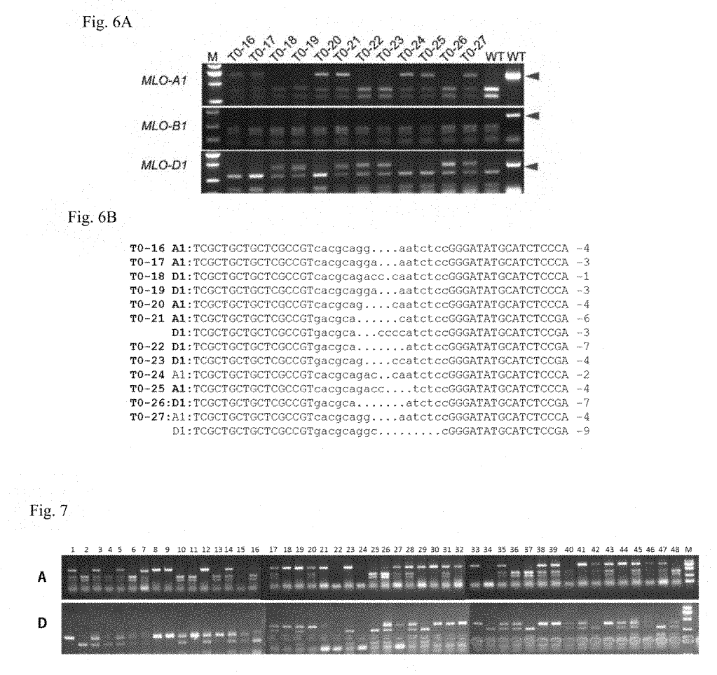

The results indicates that, after transient expression of the TALENs plasmid T-MLO into wheat immature embryo, site-specific modifications of wheat-originated MLO gene occurred in T0 plant regenerated from the immature embryo, which include heterozygous plants that merely have site-specific modification in TaMLO-A gene, heterozygous plants that merely have site-specific modification in TaMLO-D gene, heterozygous plants that have site-specific modification in both TaMLO-A gene and TaMLO-D gene (Genomic DNA was extracted from some of the heterozygous plants, and was also digested by AvaII enzyme; the results can be seen in a of FIG. 6, some of the sequencing results can be seen in b) of FIG. 6).

II. Mutant Obtained by Particle Bombardment Transient Expression of TALENs can be Stably Transmitted to the Progeny

T1 plants were obtained through self-fertilization of the T0 mutant obtained by the above particle bombardment transient expression of T-MLO. Specific primers were used to respectively amplify the TaMLO-A gene, TaMLO-B gene and TaMLO-D gene through PCR, and the PCR products were the digested by a single enzyme AvaII (please refer to step I for specific steps). The mutations of T1 plants were examined. For example, the genetype of T0-21 was AaBBDd, 48 progeny were obtained from T1 population. Regarding A, 13 plants were AA, 26 plants were Aa, 9 plants were aa; regarding D, 9 plants were DD, 24 plants were Dd, 15 plants were dd, which substantially complied with Mendelian inheritance (FIG. 7). This indicates that mutant obtained by particle bombardment transient transformation of TALENs can stably transmit the mutation to the progeny.

III. Using PCR Methods to Detect Whether the T0 and T1 Plants Contain the Vector T-MLO

In T-MLO vector, the TALEN is initiated by a maize promoter Ubi-1. A primer pair was designed according to Ubi-1, which was used to amplify T0 plants and T1 plants, so as to detect whether the genome of a mutant obtained by particle bombardment transient transformation will comprise the integrated TALENs vector.

Ubi-F:
5′-CAGTTAGACATGGTCTAAAGGACAATTGAG-3′;
Ubi-R:
5′-CCAACCACACCACATCATCACAACCAA-3′.

Theoretically, the amplified fragment should be about 1387 bp, and the sequence should be positions 191-1577 of SEQ ID NO:6. SEQ ID NO:6 is the whole sequence of the TALENs (T-MLO).

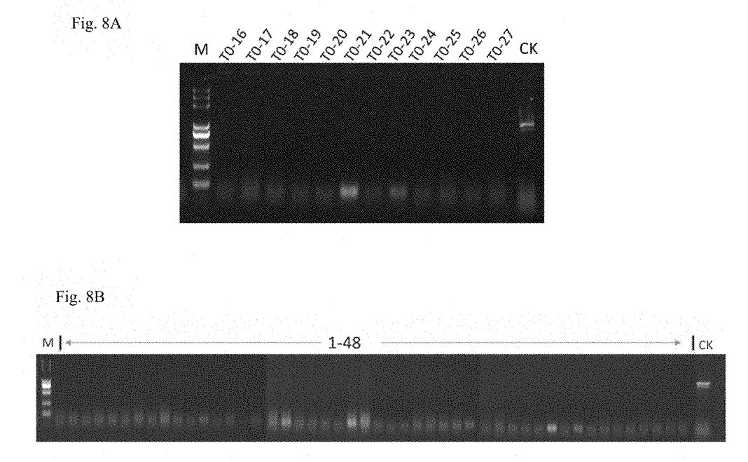

The results indicate that, none of the T0 plants can be amplified the target band (a) of FIG. 8). As for T1 population, similarly the progeny of T0-14 was selected for amplification, and it can be seen that none of the 48 progeny plants can be amplified the target band (b of FIG. 8), This indicates that, the present invention prevents the insertion or carrying of a transgene when performing site-specific modification in a plant, and the mutant as obtained have relatively high bio-safety and can be stably transmitted.

### Example 3. Further Verification of the Transient Expression-Based Gene Editing Approach

The genome editing approach of the invention was further tested using five different wheat genes as targets.

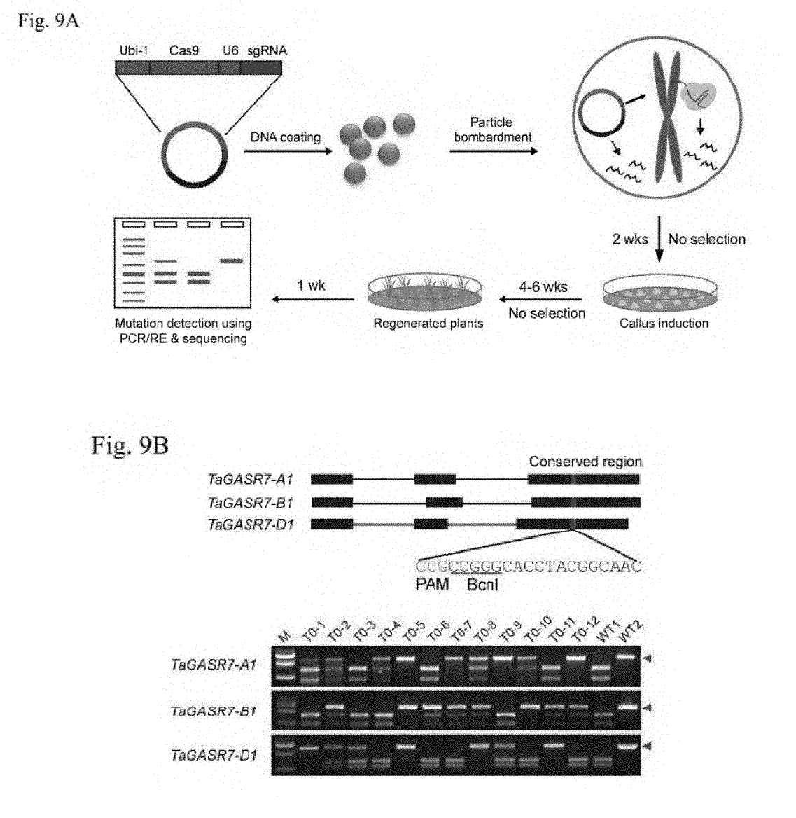

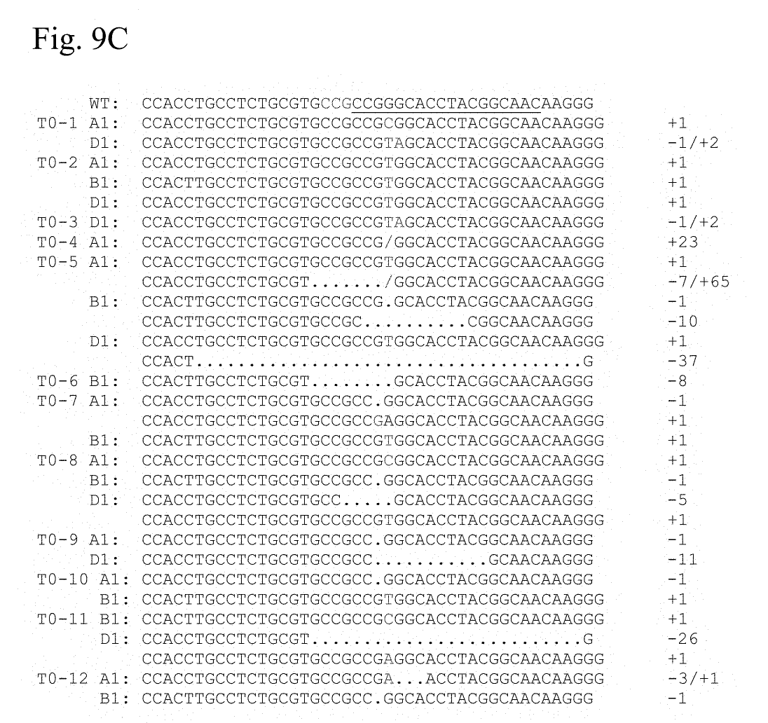

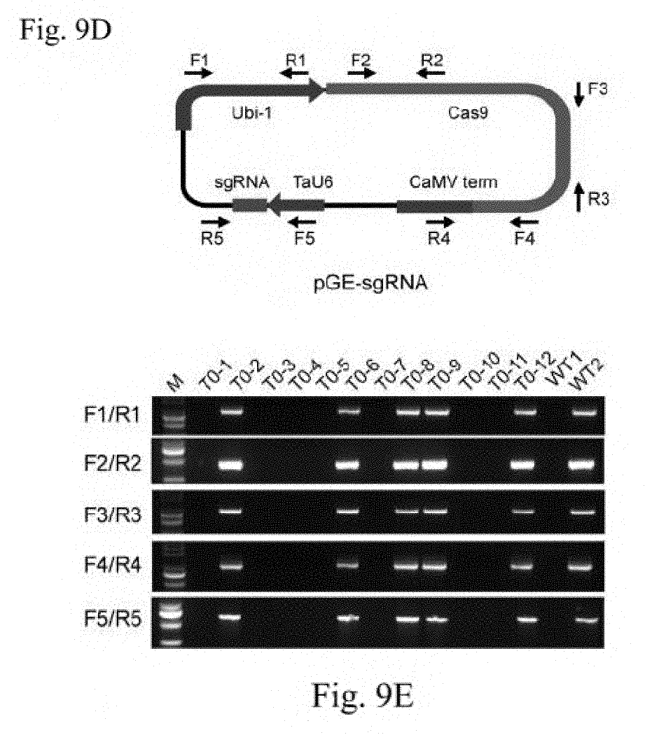

First, three homoeologs of TaGASR7 (TaGASR7 A1, TaGASR7-R1 and TaGASR7 D1), which are known as involved in determining grain length and weight¹, were edited. The three homeologs each have three exons and two introns (FIG. 9B). sgRNAs that target exon 3 were designed because this exon is highly conserved. After initial testing of nuclease activity in protoplasts², the most effective sgRNA expression cassette (Table 5) was combined with Cas9 in a single construct (pGE-TaGASR7, FIG. 9D). This was introduced by particle bombardment into immature embryos of two common wheat varieties (Bobwhite and Kenong199). Embryogenic calli developed in 2 weeks, and a large number of seedlings (2-3 cm high) were regenerated from the calli in 4-6 weeks. In contrast with most plant genome editing experiments, no herbicide or antibiotics was added to the medium to select for transgenic plants (FIG. 9A). Under the selection-free conditions, the total time for seedling regeneration was 6-8 weeks, which is 2-4 weeks shorter than that published in previous studies³.

The sgRNA target site in the regenerated T0 seedlings was analyzed by PCR-RE, first using a conserved primer set (Table 6) that recognizes all three TaGASR7 homoeologs and then with three primer pairs specific for the three respective homoeologs (Table 6). A total of 80 TaGASR7 mutants with indels in the targeted region were identified among 1005 (8.0%) Bobwhite seedlings, and another set of 21 such mutants among 283 (7.4%) Kenong 199 seedlings (Table 7). Targeted mutations were observed in all three homoeologs (FIG. 9b, 9c). Among the 80 Bobwhite mutant seedlings, nearly all combinations of TaGASR7-A1, TaGASR7-B1 and TaGASR7 D1 mutants were identified, including 51 mutants with at least one allele modified in all three genomes (Table 8). Eight of these 51 mutants had all six alleles simultaneously knocked out (Table 8). These data suggest that the method of the invention is highly efficient in generating targeted mutations of TaGASR7 in T0 populations.

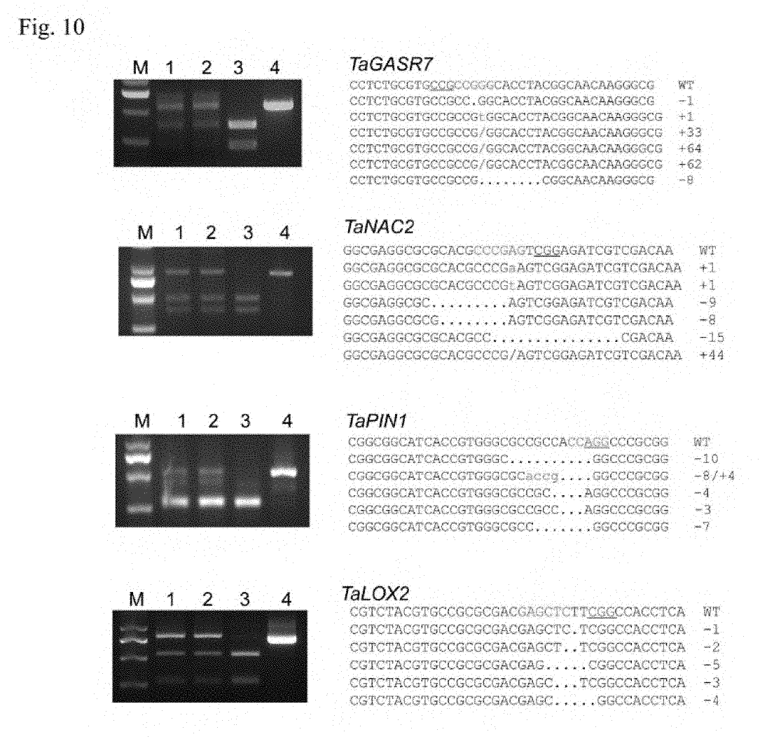

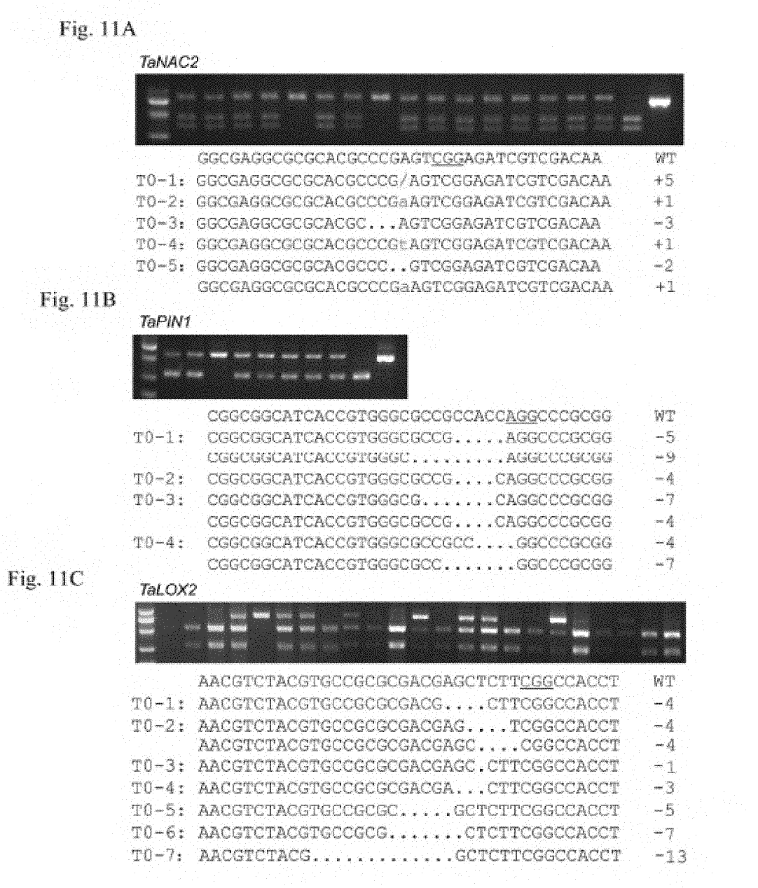

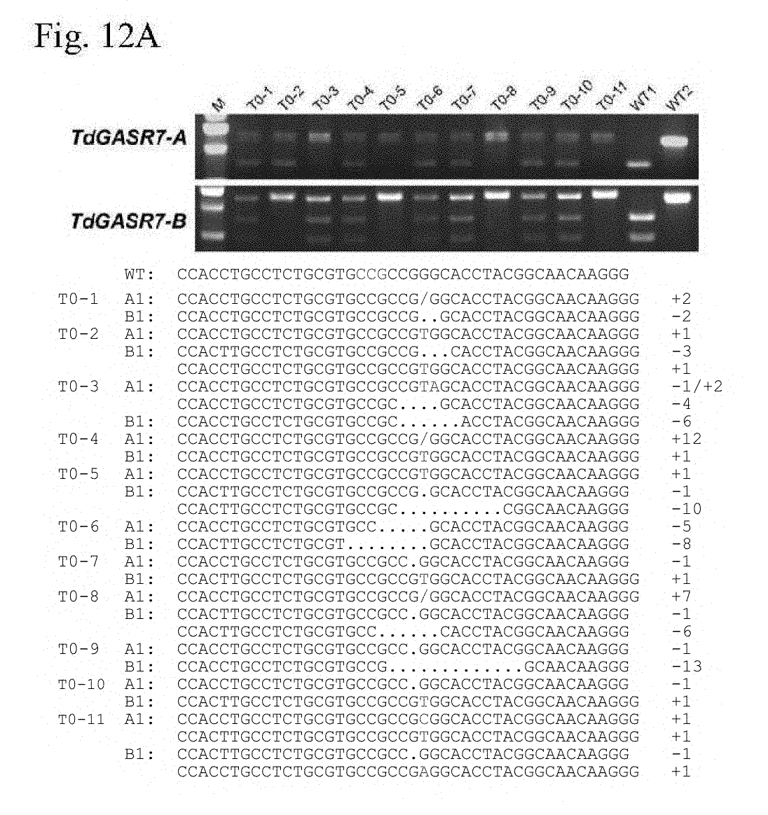

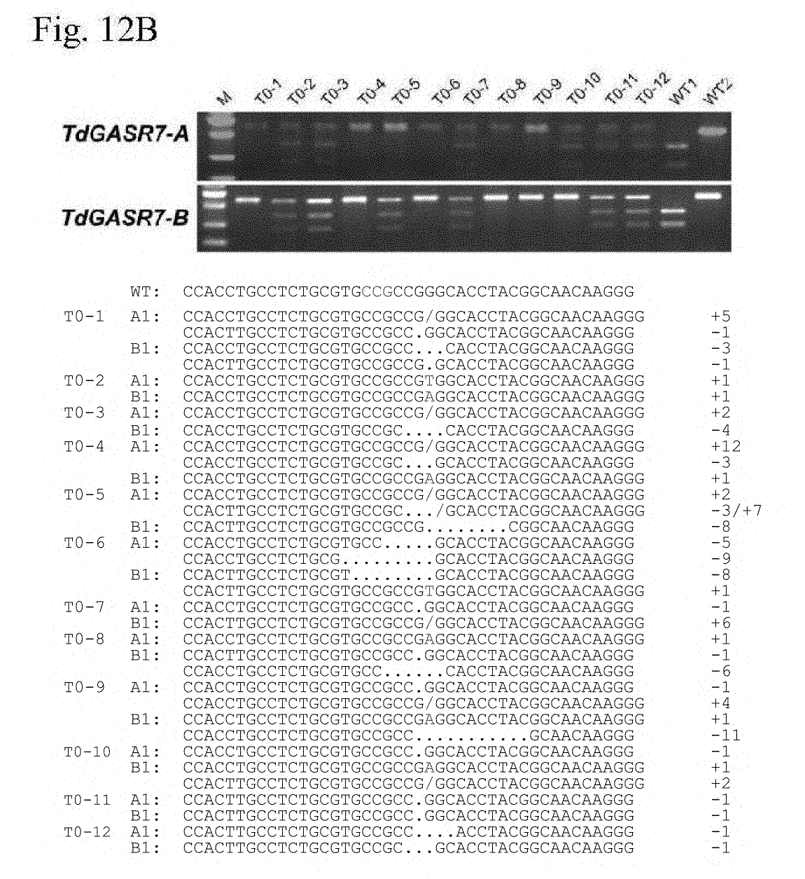

Next, other wheat genes were targeted to determine if the approach was generally applicable. Wheat homologs of rice NAC2 and PIN1 and a wheat lipoxygenase gene (TaLOX2) were targeted. In rice, NAC2 has been found to regulate shoot branching⁴, and PIN1 is required for auxin-dependent adventitious root emergence and tillering⁵. TaLOX2 is highly expressed during grain development and may affect the storability of wheat grains⁶. CRISPR constructs were developed for each of the four genes (FIG. 10 and Table 5), and a large number of T0 seedlings were obtained by transient expression approach (FIG. 9a, Table 7). For simplicity, only conserved primers (Table 6) were designed to detect mutations in TaNAC2, TaPIN1, and TaLOX2, the latter of which exists as a single copy (TaLOX2 D1 in the D genome). Targeted mutations in all three genes were easily identified in the T0 seedlings by PCR-RE analysis (FIG. 11). The mutation frequency varied from 2.5% to 9.2%, and we identified 34 talox2-dd homozygous mutants among 76 mutant plants (44.7%) (Table 7). In addition to common wheat, durum wheat (Triticumturgidum L. var. durum, AABB, 2n=4x=28) is also an important crop widely used for pasta foods. Because GASR7 is highly conserved in tetraploid and hexaploid wheat, we introduced vector pGE-TaGASR7 into two different durum wheat varieties. The frequency of targeted mutations in T0 seedlings of these tetraploid wheat lines exceeded 3%, and homozygous mutants resulting from simultaneous editing of all four alleles could be obtained (Table 7 and FIG. 12). These results indicate that the genome editing approach of the invention is likely to be effective for any wheat gene and wheat variety.

Because the T0 seedlings were regenerated in the absence of selection, there was a high probability that the CRISPR construct would not be integrated into the wheat genome. This was examined by testing for the presence of plasmid DNA carrying the CRISPR construct in the T0 seedlings using PCR. Primer sets (Table 6) specific for five discrete regions of each of the constructs were designed, representing all their major components (FIG. 9D). Based on this type of PCR analysis, the CRISPR construct was found to be absent in 43.8% (cv Bobwhite) (FIG. 9E) and 61.9% (cv Kenong199) of the T0 mutants for TaGASR7 (Table 7). For the other three genes, the frequencies of transgene-free seedlings were 75.0% (TaNAC2), 62.5% (TaPIN1) and 86.8% (TaLOX2) (Table 7). Likewise, absence of CRISPR construct integration was found in 54.5% to 58.3% of the T0 mutant seedlings of the two durum wheat varieties (Table 7). Thus, using this genome editing approach, it is possible to obtain targeted mutants that are free of CRISPR constructs.

It is also found that the present system could be used with other sequence-specific nucleases, such as ZFNs and TALENs. The present inventors previously described a pair of TALENs that target the MLO loci in common wheat, and reported an editing efficiency of 3.4% for seedlings regenerated on medium containing the herbicide phosphinotricin (PPT) to select for presence of the TALEN construct³. In the present work, the same pair of TALENs was delivered to immature embryos allowing the seedlings to regenerate without selection. Of 200 regenerated T0 seedlings, 13 (6.5%) carried targeted mutations, and all were transgene-free as assessed by PCR (Table 5 and Table 7).

To investigate whether the mutations produced by the method of the invention can be transmitted to the next generation, representative T0 TaGASR7, TaMLO and TaLOX2 mutants were self-pollinated, and T1 progeny were analyzed by PCR-RE. For homozygous mutations detected in T0 (including those with simultaneous editing of all six alleles), transmission rates were 100%; for the majority of the heterozygous mutants, Mendelian segregation occurred (homozygote/heterozygote/wildtype: 1:2:1) (Table 9). As anticipated, no integrated CRISPR or TALEN constructs were detected in the T1 progeny of transgene-free T0 parents (Table 9).

In summary, the SSN transient expression method of the invention offers several advantages over commonly used genome editing approaches that involve a transgenic intermediate. First, targeted gene editing occurs at a high frequency, and it is possible to quickly obtain homozygous, transgene-free mutants. The previous studies reported that sgRNA/Cas9 cassette and TALENs that integrated in the plant genomes retain their activity and can generate new mutations in the offspring^{7,3}; transgene-free mutants should reduce complexity of subsequent analysis and off-target risk. They should also be subjected to less regulatory scrutiny. Second, mutants from plants that are hard to transform can be easily obtained by the approach of the invention because plant regeneration from callus cells is possible in most species. The method of the invention may also be useful for modifying genes in vegetatively propagated crops such as potato, cassava and banana, where it is difficult or impossible to segregate away transgenes. The approach described here will accelerate the understanding of plant gene function and enable production of valuable new crop cultivars.

TABLE 5
SSN target loci and sequences.

| | | | | Detection |
|---|---|---|---|---|
| SSN ID | Target site | Oligo-F | Oligo-R | method |
| sg- | CCGCCGGGCACCTACG | CTTGTTGCCGTAGG | AAACCCGGGCACCT | PCR/RE |
| GASR7 | GCAAC | TGCCCGG | ACGGCAA | BcnI |
| sg- | CGAGGCGCGCACGCCC | CTTGCGAGGCGCGC | AAACACTCGGGCGTG | PCR/RE |
| NAC2 | GAGTCGG | ACGCCCGAGT | CGCGCCTCG | AvaI |
| sg-PIN1 | TCACCGTGGGCGCCGC | CTTGTCACCGTGGG | AAACGGTGGCGGCGC | PCR/RE |
| | CACCAGG | CGCCGCCACC | CCACGGTGA | MvaI |
| sg- | GTGCCGCGCGACGAGC | CTTGTGCCGCGCGA | AAACAAGAGCTCGTC | PCR/RE |
| LOX2 | TCTTCGG | CGAGCTCTT | GCGCGGCA | SacI |
| T-MLO | TCGCTGCTGCTCGCCGT | | | PCR/RE |
| | gacgcaggaccccatctcCGGGA | | | AvaII |
| | TATGCATCTCCGA | | | |

TABLE 6
PCR primers and their applications.

| Primer name | Primer sequence | Application |
|---|---|---|
| F1 | CAGTTAGACATGGTCTAAAGGACAATTGAG | Detecting CRISPR/TALEN |
| R1 | CCAACCACACCACATCATCACAACCAA | construct |
| F2 | CCTAAGAAGAAGAGAAAGGTCG | Detecting CRISPR construct |
| R2 | GCAGATGATAGATTGTGGGGTA | |
| F3 | GCCCATCTCTTCGATGACAAGGTTATG | Detecting CRISPR construct |
| R3 | CTTCGCAGTGGCCTTGCCAATTTC | |
| F4 | GGTGGCTTACTCTGTCCTGGTT | Detecting CRISPR construct |
| R4 | TTCCTTGTCTTCCTCCTTCCTT | |
| F5 | AGCCCGTTATTCTGACAGTTCTGGTGC | Detecting CRISPR construct |
| R5 | GTGAGCGCAACGCAATTAATGTGAG | |
| F6 | CTTAAGATTGAATCCTGTTGCCGGTC | Detecting TALEN construct |
| R6 | TCGTGCACACAGCCCAGCTTGG | |
| U6-SpeI-F | CGGACTAGTGACCAAGCCCGTTATTCTGAC | Amplifying the fragment of |
| sgRNA- | CGGACTAGTAAAAAAAGCACCGACTCGGTG | TaU6-sgRNA |
| SpeI-R | CCAC | |
| GASR7-F | GGAGGTGATGGGAGGTGGGGG | Amplifying the TaGASR7 target |
| GASR7-R | CTGGGAGGGCAATTCACATGCCA | site |
| GASR7- | CCTTCATCCTTCAGCCATGCAT | Amplifying the TaGASR7 target |
| A1/B1/D1-F | | site |
| GASR7-A1-R | CCACTAAATGCCTATCACATACG | Amplifying the TaGASR7-A1 |
| | | target site |
| GASR7-B1-R | AGGGCAATTCACATGCCACTGAT | Amplifying the TaGASR7-B1 |
| | | target site |
| GASR7-D1-R | CCTCCATTTTTCCACATCTTAGTCC | Amplifying the TaGASR7-D1 |
| | | target site |
| NAC2-F | GGGATCAAGAAGGCCCTGGTGTTT | Amplifying the TaNAC2 target site |
| NAC2-R | TCGATCTCGGTATCTGACGGTCTGTG | |
| PIN1-F | GATGGACTTCATGATGATCGCC | Amplifying the TaPIN1 target site |
| PIN1-R | AACGGCACCGAGGCGTACAACGA | |
| LOX2-F | CGTCTACCGCTACGACGTCTACAACG | Amplifying the TaLOX2 target site |
| LOX2-R | GGTCGCCGTACTTGCTCGGATCAAGT | |
| MLO-A1-F | TGGCGCTGGTCTTCGCCGTCATGATCATCGTC | Amplifying the TaMLO-A1 target |
| MLO-A1-R | TACGATGAGCGCCACCTTGCCCGGGAA | site |
| MLO-B1-F | ATAAGCTCGGCCATGTAAGTTCCTTCCCGG | Amplifying the TaMLO-B1 target |
| MLO-B1-R | CCGGCCGGAATTTGTTTGTGTTTTTGTT | site |
| MLO-D1-F | TGGCTTCCTCTGCTCCCTTGGTGCACCT | Amplifying the TaMLO-D1 target |
| MLO-D1-R | TGGAGCTGGTGCAAGCTGCCCGTGGACATT | site |

TABLE 7
Transgene-free genome editing in wheat by transient expression of sequence-
specific nucleases.

| | | | | | No. of | |
|---|---|---|---|---|---|---|
| | | | | No. of | homozygous | |
| | | | No. of | mutants/ | mutants/ | No. of transgene-free |
| | | Wheat | regenerated | Mutagenesis | Frequency | mutants/ |
| SSN | Gene | variety | plants | (%)^{a} | (%)^{b} | Frequency (%)^{c} |
| CRISPR/Cas | TaGASR7 | Bobwhite | 1005 | 80 (8.0) | 8 (10.0) | 35 (43.8) |
| | TaGASR7 | Kenong199 | 283 | 21 (7.4) | 4 (19.0) | 13 (61.9) |
| | TaNAC2 | Kenong199 | 394 | 16 (4.1) | N.D | 12 (75.0) |
| | TaPIN1 | Kenong199 | 317 | 8 (2.5) | N.D | 5 (62.5) |
| | TaLOX2 | Kenong199 | 824 | 76 (9.2) | 34 (44.7) | 66 (86.8) |
| | TdGASR7 | Shimai11 | 334 | 11 (3.3) | 3 (27.3) | 6 (54.5) |
| | TdGASR7 | Yumai4 | 356 | 12 (3.4) | 5 (41.7) | 7 (58.3) |
| TALEN | TaMLO | Bobwhite | 200 | 13 (6.5) | 0 | 13 (100) |

TABLE 8
Genotypes of the 80 T0 tagasr7 mutants with respect to mutations in the
TaGASR7-A1, TaGASR7-B1 and TaGASR7-D1 homeoalleles.

| Genotype of | | | | Genotype of | | | |
|---|---|---|---|---|---|---|---|
| TaGASR7 | Plant | Mutation detected | SSN- | TaGASR7 | Plant | Mutation detected | SSN- |
| homoeologs | ID | (bp)^{a} | free | homoeologs | ID | (bp)^{a} | free |
| AaBBDD | T0-4 | +23(Aa) | YES | AaBbdd | T0-8 | +1(Aa); −1(Bb); −5, | NO |
| | | | | | | +1(dd) | |
| | T0-28 | N.D. | NO | | T0-52 | N.D. | YES |
| | T0-36 | +12(Aa) | YES | | T0-58 | N.D. | NO |
| aaBBDD | N.A. | | | | T0-66 | N.D. | YES |
| AABbDD | T0-6 | −8(Bb) | NO | AabbDd | T0-31 | +1(Aa); −4/+1, +1(bb); | YES |
| | | | | | | +8(Dd) | |
| | T0-49 | +123(Bb) | YES | | T0-33 | N.D. | NO |
| AAbbDD | N.A. | | | | T0-50 | N.D. | NO |
| AABBDd | T0-3 | −1/+2(Dd) | YES | | T0-55 | N.D. | NO |
| | T0-30 | N.D. | NO | | T0-69 | N.D. | YES |
| | T0-56 | N.D. | NO | | T0-76 | N.D. | NO |
| | T0-79 | +1(Dd) | YES | | T0-78 | N.D. | NO |
| AABBdd | T0-71 | +63, −1(dd) | YES | Aabbdd | T0-16 | −6/+1(Aa); −25(bb); | YES |
| | | | | | | +1, −26(dd) | |
| AaBbDD | T0-15 | +82(Aa); −1(Bb) | NO | | T0-23 | −4(Aa); +1, +89(bb) | NO |
| | | | | | | −6, +1(dd) | |
| | T0-44 | N.D. | YES | | T0-38 | N.D. | NO |
| | T0-60 | N.D. | NO | | T0-40 | N.D. | NO |
| AabbDD | T0-10 | −1(Aa); +1(bb) | YES | | T0-45 | N.D. | YES |
| | T0-14 | −1(Aa); | NO | | T0-68 | N.D. | YES |
| aaRbDD | T0-7 | −1, +1(aa); +1(Bb) | YES | aaRbDd | T0-19 | +129, +1(aa); | NO |
| | | | | | | −2/+1(Bb); −16(Dd) | |
| | T0-12 | −3/+1(aa); −1(Bb) | NO | | T0-32 | N.D. | NO |
| AaBBDd | T0-35 | +1(Aa); +1(Dd) | NO | | T0-57 | N.D. | NO |
| | T0-51 | | YES | | T0-59 | N.D. | NO |
| | T0-61 | +2(Aa); −6(Dd) | YES | | T0-64 | N.D. | NO |
| | T0-77 | N.D. | YES | aaRbdd | T0-17 | −2, −3(aa); −1(Bb); −10, | NO |
| | | | | | | −7(dd) | |
| AaBBdd | T0-1 | +1 (Aa); −1/ | YES | | T0-26 | N.D. | NO |
| | | +2 (dd) | | | | | |
| aaRBDd | T0-9 | −1(aa); −11(Dd) | NO | | T0-41 | +8, −1(aa); +24(Bb); | YES |
| | | | | | | +25, +1(dd) | |
| aaBBdd | N.A. | | | | T0-54 | N.D. | YES |
| aabbDD | N.A. | | | | T0-62 | N.D. | NO |
| AABbDd | T0-22 | +1(Bb); −3 (Dd) | NO | | T0-70 | N.D. | NO |
| | T0-24 | −7(Bb); +82(Dd) | NO | aabbDd | T0-13 | +1(aa); +1, −12(bb); | NO |
| | | | | | | −1(Dd) | |
| | T0-42 | N.D. | YES | | T0-37 | +3, −1(aa); +1, | YES |
| | | | | | | −2/+1(bb); −2(Dd) | |
| | T0-46 | N.D. | NO | | T0-43 | N.D. | YES |
| | T0-74 | N.D. | NO | | T0-63 | N.D. | NO |
| AABbdd | T0-11 | +1(Bb); −26, | YES | | T0-80 | N.D. | YES |
| | | +1(dd) | | | | | |
| AAbbDd | N.A. | | | AaBbDd | T0-2 | +1(Aa); +1(Bb); | NO |
| | | | | | | +1(Dd) | |
| AAbbdd | N.A. | | | | T0-18 | −4/+1(Aa); +2(Bb); | NO |
| | | | | | | +1(Dd) | |
| aabbdd | T0-5 | +1, −7/+65(aa); −1, −10 | YES | | T0-20 | −6(Aa); −17(Bb); | YES |
| | | 10(bb); +1, −37(dd) | | | | +5(Dd) | |
| | T0-27 | +1(aa); −1, −7(bb); −5, | YES | | T0-21 | −5/+1(Aa); +1(Bb); +1, | NO |
| | | +1(dd) | | | | −19/+166, | |
| | | | | | | −19/+167(Dd) | |
| | T0-29 | −4/+1(aa); | YES | | T0-25 | +1(Aa); −9(Bb); | NO |
| | | +63, +1(bb); | | | | −1(Dd) | |
| | | +1, −1(dd) | | | | | |
| | T0-39 | N.D. | NO | | T0-34 | N.D. | NO |
| | T0-47 | N.D. | NO | | T0-48 | N.D. | YES |
| | T0-65 | N.D. | YES | | T0-53 | N.D. | YES |
| | T0-73 | N.D. | NO | | T0-67 | N.D. | NO |
| | T0-75 | N.D. | NO | | T0-72 | N.D. | YES |

N.A., not available. These mutant types were not obtained from the experiments;
N.D., not detected.
^{a}“−” indicates deletion of the indicated number of nucleotides; “+” indicates insertion of the indicated number of nucleotides; “−/+” indicates simultaneous deletion and insertion of the indicated number of nucleotides at the same site.

TABLE 9
Molecular and genetic analysis of SSN-induced mutations in TaGASR7,
TaMLO and TaLOX2 homoeologs and their transmission to the T1 generation.

| | | | Analysis of T0 plants | | | Segregation of mutations in the T1 generation | | | | | |
|---|---|---|---|---|---|---|---|---|---|---|---|
| | | | Genotype | Mutation | | No. of | | | | Mutation | SSN- |
| SSN | Wheat | Plant | of | detected | SSN- | tested | Wild | | | Transmission | free |
| ID | variety | ID | homeologs | (bp)^{a} | free | plants | type | Hetero | Homo | (%)^{b} | (%)^{c} |
| sg- | Bob- | T0-1 | Aa | +1 | YES | 26 | 7 | 13 | 6 | 73.1^{d} | 100 |
| GASR7 | white | | | | | | (AA) | (Aa) | (aa) | | |
| | | | BB | | | | 26 | 0 | 0 | | |
| | | | | | | | (BB) | (Bb) | (bb) | | |
| | | | dd | −1/+2 | | | 0 | 0 | 26 | 100 | |
| | | | | | | | (DD) | (Dd) | (dd) | | |
| | | T0-5 | aa | +1, −7/ | YES | 44 | 0 | 0 | 44 | 100 | 100 |
| | | | | +65 | | | (AA) | (Aa) | (aa) | | |
| | | | bb | −1, −10 | | | 0 | 0 | 44 | 100 | |
| | | | | | | | (BB) | (Bb) | (bb) | | |
| | | | dd | +1, −37 | | | 0 | 0 | 44 | 100 | |
| | | | | | | | (DD) | (Dd) | (dd) | | |
| | | T0-10 | Aa | −1 | YES | 35 | 10 | 16 | 9 | 71.4^{d} | 100 |
| | | | | | | | (AA) | (Aa) | (aa) | | |
| | | | bb | +1 | | | 0 | 0 | 35 | 100 | |
| | | | | | | | (BB) | (Bb) | (bb) | | |
| | | | DD | | | | 35 | 0 | 0 | | |
| | | | | | | | (DD) | (Dd) | (dd) | | |
| | | T0-16 | Aa | −6/+1 | YES | 30 | 9 | 14 | 7 | 70.0^{d} | 100 |
| | | | | | | | (AA) | (Aa) | (aa) | | |
| | | | bb | −25 | | | 0 | 0 | 30 | 100 | |
| | | | | | | | (BB) | (Bb) | (bb) | | |
| | | | dd | +1, −26 | | | 0 | 0 | 30 | 100 | |
| | | | | | | | (DD) | (Dd) | (dd) | | |
| | | T0-20 | Aa | −6 | YES | 29 | 8 | 14 | 7 | 72.4^{d} | 100 |
| | | | | | | | (AA) | (Aa) | (aa) | | |
| | | | Bb | −17 | | | 7 | 15 | 7 | 75.9^{d} | |
| | | | | | | | (BB) | (Bb) | (bb) | | |
| | | | Dd | +5 | | | 8 | 15 | 6 | 72.4^{d} | |
| | | | | | | | (DD) | (Dd) | (dd) | | |
| | | T0-36 | Aa | +12 | YES | 29 | 9 | 14 | 6 | 69.0^{d} | 100 |
| | | | | | | | (AA) | (Aa) | (aa) | | |
| | | | BB | | | | 29 | 0 | 0 | | |
| | | | | | | | (BB) | (Bb) | (bb) | | |
| | | | DD | | | | 29 | 0 | 0 | | |
| | | | | | | | (DD) | (Dd) | (dd) | | |
| T- | Bob- | T0-1 | Aa | −4 | YES | 19 | 6 | 9 | 4 | 68.0^{d} | 100 |
| MLO | white | | | | | | (AA) | (Aa) | (aa) | | |
| | | | BB | | | | 19 | 0 | 0 | | |
| | | | | | | | (BB) | (Bb) | (bb) | | |
| | | | Dd | −5 | | | 5 | 10 | 4 | 73.7^{d} | |
| | | | | | | | (DD) | (Dd) | (dd) | | |
| | | T0-3 | aa | −3, +5 | YES | 35 | 0 | 0 | 35 | 100 | 100 |
| | | | | | | | (AA) | (Aa) | (aa) | | |
| | | | BB | | | | 35 | 0 | 0 | | |
| | | | | | | | (BB) | (Bb) | (bb) | | |
| | | | Dd | −2/+11 | | | 7 | 23 | 5 | 80.0^{d} | |
| | | | Dd | −2/+11 | | | (DD) | (Dd) | (dd) | | |
| | | T0-4 | AA | | YES | 28 | 28 | 0 | 0 | | 100 |
| | | | | | | | (AA) | (Aa) | (aa) | | |
| | | | Bb | −6 | | | 6 | 14 | 8 | 78.6^{d} | |
| | | | | | | | (BB) | (Bb) | (bb) | | |
| | | | Dd | −12 | | | 8 | 13 | 7 | 71.4^{d} | |
| | | | | | | | (DD) | (Dd) | (dd) | | |
| sg- | Kenong | T0-3 | Dd | −1 | YES | 23 | 5 | 14 | 4 | 78.3^{d} | 100 |
| | | | | | | | (DD) | (Dd) | (dd) | | |
| | | T0-16 | Dd | −7 | YES | 42 | 8 (DD) | 24 | 10 | 81.0^{d} | 100 |
| | | | | | | | | (Dd) | (dd) | | |
| | | T0-22 | dd | −3, −5 | YES | 30 | 0 (DD) | 0 (Dd) | 30 | 100 | 100 |
| | | | | | | | | | (dd) | | |
| | | T0-35 | dd | −2, −9 | YES | 52 | 0 (DD) | 0 (Dd) | 52 | 100 | 100 |
| | | | | | | | | | (dd) | | |
| | | T0-65 | dd | +7, −10 | YES | 18 | 0 (DD) | 0 (Dd) | 18 | 100 | 100 |
| | | | | | | | | | (dd) | | |
| | | T0-68 | Dd | −11 | YES | 28 | 7 (DD) | 16 | 5 (dd) | 75.0^{d} | 100 |
| | | | | | | | | (Dd) | | | |

Hetero, heterozygous;
Homo, homozygous.
^{a}“−” indicates deletion of the indicated number of nucleotides; “+” indicates insertion of the indicated number of nucleotides; “−/+” indicates simultaneous deletion and insertion of the indicated number of nucleotides at the same site.
^{b}Based on the number of plants carrying the observed mutation over the total number of plants tested.
^{c}Based on the number of mutant plants not harboring the intact CRISPR and TALEN construct over the total number of mutant plants tested.
^{d}Segregation of the heterozygous lines conforms to a Mendelian 1:2:1 ratio according to the χ2 test (P > 0.5).

### General Methods

Selection of sgRNA Targets

Several sgRNA targets for each gene were designed in the conserved domains of the A, B, and D genomes of wheat. The activities of the sgRNAs were evaluated by transforming pJIT163-Ubi-Cas9 plasmid³ and TaU6-sgRNA plasmid⁸ into wheat protoplasts. Total genomic DNA was extracted from the transformed protoplasts and fragments surrounding the targeted sequences were amplified by PCR. The PCR-RE digestion screen assay was used to detect sgRNA activity′ (FIG. 9).

### Protoplast Assays

Spring wheat variety Bobwhite and winter wheat variety Kenong199 were used in this study. Wheat protoplasts transformation was performed as described⁸.

Construction of pGE-sgRNA Vectors

Fragments of active TaU6-sgRNA (Table 5) were amplified from TaU6-sgRNA plasmid⁸ using the primer set U6-SpeI-F/sgRNA-SpeI-R with a SpeI restriction site (Table 6). The PCR products were digested with SpeI and inserted into SpeI-digested pJIT163-Ubi-Cas9 (ref 3) to yield the fused expression vector pGE-sgRNA (FIG. 9D).

### Biolistic Transformation of Wheat by the Transient Expression System

Biolistic transformation was performed as previously described⁹. Plasmid DNA (pGE-sgRNA or T-MLO³) (FIG. 9D) was used to bombard wheat embryos. After bombardment, embryos were transferred to callus induction medium. In the 3rd week all calli were transferred to regeneration medium. After 3-5 weeks, sprouts appeared on the surface of the calli. These were transferred to rooting medium, and a large number of T0 seedlings were obtained about 1 week later. No selective agents were used in any part of the tissue culture process (FIG. 9A).

### Accession Codes.

Sequence data are available with NCBI Genbank under accession numbers KJ000052 (TaGASR7-A1), KJ000053 (TaGASR7-B1), KJ000054 (TaGASR7-D1), AY625683 (TaNAC2), AY496058 (TaPIN1) and GU167921 (TaLOX2).

### REFERENCES

1. Ling, H. et al. Nature. 496, 87-90 (2013).
2. Shan, Q. et al. Nat. Protoc. 9, 2395-2410 (2014).
3. Wang, Y. et al. Nat. Biotechnot 32, 947-95 (2014).
4. Mao, C. et al. New Phytol. 176, 288-298 (2007).
5. Xu, M. et al. Plant Cell Physiol. 46, 1674-1681 (2005).
6. Feng, B. et al. J. Cereal Sci. 52, 387-394 (2010).
7. Lawrenson, T. et al. Genome Biol. 16, 258 (2015).
8. Shan, Q. et al. Nat. Biotechnol. 31, 686-688 (2013).
9. Zhang, K. et al. J. Genet. Genomics. 42, 39-42 (2015).

1. A method comprising utilizing a transient expression system for conducting site-specific modification to a target site of a target gene in a plant.

2. A method for conducting site-specific modification to a target site of a target gene in a plant of interest, comprising the following steps:
transiently expressing a sequence-specific nuclease in a cell or tissue of the plant, wherein said sequence-specific nuclease is specific to the target site and the target site is cleaved by said nuclease; thereby site-specific modification of the target site is achieved through DNA repairing of the plant.

3. The method of claim 2, wherein the process for transiently expressing the sequence-specific nuclease in a cell or tissue of the plant comprises the following steps:
a) introducing a genetic material for expressing the sequence-specific nuclease into the cell or tissue of the plant; and
b) culturing the cell or tissue obtained in step a) in the absence of selection pressure, wherein the sequence-specific nuclease is transiently expressed in the cell or tissue of the plant of interest and the genetic material not integrated into the genome of the plant is degraded.

4. The method of claim 2, wherein the genetic material is a recombinant vector such as a DNA plasmid, a DNA linear fragment, or an RNA.

5. The method of claim 2, wherein said sequence-specific nuclease is a CRISPR/Cas9 nuclease, a TALENs nuclease, a Zinc finger nuclease, or any nuclease that can achieve genome editing.

6. The method of claim 5, where the sequence-specific nuclease is a CRISPR/Cas9 nuclease, the genetic material for expressing the CRISPR/Cas9 nuclease specific to the target fragment gene is composed of
a) a recombinant vector or DNA fragment for transcribing a guide RNA and for expressing Cas9 protein;
b) two recombinant vectors or DNA fragments for transcribing crRNA and tracrRNA respectively and for expressing Cas9 protein;
c) a recombinant vector or DNA fragment for transcribing a guide RNA;
d) two recombinant vectors or DNA fragments for transcribing crRNA and tracrRNA respectively and a recombinant vector or DNA fragment or RNA for expressing Cas9 protein; or
e) a guide RNA, or both a crRNA and a tracrRNA, and a recombinant vector or DNA fragment or RNA for expressing Cas9 protein,
wherein the guide RNA is an RNA with a palindromic structure which is formed by partial base-pairing between the crRNA and tracrRNA; and wherein the crRNA contains an RNA fragment that can complementarily binding to the target site.

7. The method of claim 5, where the sequence-specific nuclease is a TALENs nuclease, the genetic material for expressing the nuclease specific to the target fragment is a recombinant vector or DNA fragment or RNA that expresses paired TALEN proteins, and wherein the TALEN protein is composed of a DNA binding domain capable of recognizing and binding to the target site, and a Fok I domain.

8. The method of claim 5, where the sequence-specific nuclease is a Zinc finger nuclease, the genetic material for expressing the nuclease specific to the target site is a recombinant vector DNA fragment or RNA that expresses paired ZFN proteins, and wherein the ZFN protein is composed of a DNA binding domain capable of recognizing and binding to the target site, and a Fok I domain.

9. The method of claim 2, wherein the cell is any cell that can act as a transient expression recipient and regenerate into a whole plant through tissue culture; the tissue is any tissue that can act as a transient expression recipient and regenerate into a whole plant through tissue culture.

10. The method of claim 9, wherein the cell is a protoplast cell or suspension cell; the tissue is a callus tissue, immature embryo, mature embryo, leaf, shoot apex, hypocotyl, or young spike.

11. The method of claim 2, wherein the approach for delivery of the genetic material is particle bombardment, Agrobacterium-mediated transformation, PEG-mediated protoplast transformation, electrode transformation, silicon carbide fiber-mediated transformation, vacuum infiltration transformation, or any other genetic transformation approach.

12. The method of claim 2, wherein the site-specific modification is an insertion, deletion, and/or replacement mutation in the target site.

13. A cell or tissue, wherein the cell or tissue is obtained by using the method of claim 2 to conduct site-specific modification to a target site of a target gene in a plant of interest so as to allow the target gene to lose its functions or gain a function.

14. A modified plant, characterized in that: the modified plant is obtained by culturing the cell or tissue of claim 13; or
a transgene-free modified plant, wherein the transgene-free modified plant is obtained from said modified plant and contains no integrated exogenous gene in the genome and is genetically stable.

15. A method for breeding transgene-free modified plant, comprising the following steps:
(a) performing site-specific modification to a target site of a target gene in a plant of interest using the method of claim 2, so as to obtain a modified plant;
(b) screening for a plant from the modified plants obtained in step (a), wherein the functions of the target gene in said plant are lost or change, the genome of said plant is free of integrated exogenous gene, and said plant is genetically stable.
